(19) United States
(12) Patent Application Publication
(10) Pub. No.: US 2026/0283526 A1
(43) Pub. Date: Sep. 24, 2026

(54) SYSTEMS FOR ELECTROENCEPHALOGRAPY AND METHODS FOR MANUFACTURE OF THE SAME

(71) Applicant: The General Hospital Corporation, Boston, MA (US)

(72) Inventor: Giorgio Bonmassar, Boston, MA (US)

(21) Appl. No.: 18/881,222

(22) PCT Filed: Jul. 6, 2023
(86) PCT No.: PCT/US2023/069706

§ 371 Date: Jan. 3, 2025

Related U.S. Application Data
(60) Provisional application No. 63/367,811, filed on Jul. 6, 2022.

(51) Int. Cl. A61B 5/291; A61B 5/265; A61B 5/273

(52) U.S. Cl. CPC A61B 5/291; A61B 5/265; A61B 5/273; A61B 2562/125; A61B 2562/222

(57) ABSTRACT

Systems and methods are provided for forming a system for transmitting electrical signals to or from the head of a subject. The system includes a base layer. A layer of conductive, non-ferrous material may be deposited along an upper surface of the base layer using a thin-film deposition technique, which is patterned into a conductive trace that extends between a proximal end and a distal end of the base layer. A thick film forming an electrode is positioned relative to a distal end of the conductive trace.

### CROSS-REFERENCE TO RELATED APPLICATIONS

This application is based on, claims priority to, and incorporates herein by reference for all purposes, U.S. Provisional Application Ser. No. 63/367,811, filed Jul. 6, 2022, and entitled, “SYSTEMS FOR ELECTROENCEPHALOGRAPHY AND METHODS FOR MANUFACTURE OF THE SAME.”

### BACKGROUND

Electrophysiological brain signals are typically recorded by an electroencephalogram (“EEG”) system. The EEG system may be a device that measures the electrical activity in the brain via a multitude of electrodes attached to a patient's scalp by way of a cap or a special glue or paste and connected to the EEG system through wires called leads. The electrodes detect the electrophysiological signals, and the EEG system amplifies and records them onto paper or a computer for analysis by medical personnel.

Recording the EEG signals allows medical personnel to view information (e.g., a graph) reflecting the activity of thousands of neurons in the brain. The pattern of activity in the recorded EEG signals or brain waves changes with the level of the patient's arousal-if the patient is relaxed, the graph shows many slow, low-frequency brain waves; if the patient is excited, the graph shows many fast, high-frequency brain waves.

While EEG provides useful temporal information regarding the brain's electrical activity, EEG provides very low spatial resolution and cannot be used for determining the exact location of the recorded activity in the brain. However, high spatial resolution is often essential for diagnosing and treating many brain-related conditions, such as localized brain tumors and aneurysms.

Magnetic resonance imaging (“MRI”) is able to provide high anatomical special resolution. Furthermore, functional MRI (“fMRI”) can be performed using an MRI system to acquire information about the function of the brain. MRI is a technique that utilizes magnetic and radio frequency (“RF”) fields to provide high-quality image slices of the brain along with detailed metabolic and anatomical information. Radio waves 10,000-30,000 times stronger than the earth's magnetic field are transmitted through the patient's body. This affects the patient's hydrogen atoms, forcing the nuclei into a different position. As the nuclei move back into place, they send out radio waves of their own. An MRI scanner picks up those radio waves, and a computer converts them into images based on the location and strength of the incoming waves.

fMRI uses an MRI system to detect changes in cerebral blood volume, flow, and oxygenation that locally occur associated with an increased neuronal activity induced by functional paradigms. This physiological response is often referred to as the “hemodynamic response.” The hemodynamic response to neuronal activity provides a mechanism for image contrast commonly referred to as the blood-oxygen-level-dependent (BOLD) signal contrast. An MRI system can be used to acquire signals from the brain over a period of time. As the brain performs a task, these signals are modulated synchronously with task performance to reveal which brain regions are involved in completing the task. The series of fMRI time-course images must be acquired at a high enough rate to see the changes in brain activity induced by the functional paradigm. In addition, because the neuronal activity may occur at widely dispersed locations in the brain, a relatively large 3D volume or multi-slice volume must be acquired in each time frame.

In order to take advantage of the high temporal resolution of EEGs and the high spatial resolution of MRIs and fMRIs, medical personnel has been seeking ways to acquire EEG data and MRI or fMRI data simultaneously. Such simultaneous recording would provide the high spatio-temporal resolution needed to study brain activity during different tasks, such as visual, auditory, or motor tasks. Currently, no single brain imaging technology can provide the resolution needed to study this brain activity. A combination of EEGs and MRIs/fMRIs would provide the required resolution while improving the accuracy of diagnosis of many brain-related conditions.

The combination of EEG and MRI/fMRI is impractical or, at best, limited for a variety of fundamental reasons. First, the magnetic fields required for MRI require that no ferromagnetic materials be utilized near the MRI system. Beyond this fundamental challenge with material choice and sensor design, the changing magnetic and RF fields of an MRI/fMRI system can introduce significant undesirable artifacts into the EEG recordings. When EEG leads are placed inside an MRI scanner, even if dangerous currents are avoided, the rapidly changing RF fields may introduce smaller signals that can obscure the EEG signals. Further, the EEG sensors can compromise the integrity of the MRI data. That is, the presence of the EEG electrodes inside the MRI scanner and the electromagnetic radiation emitted by the EEG machine can disturb the homogeneity of the magnetic field, compromising the quality of the MRI image scans.

Even if these design and data integrity concerns are managed, fundamental safety issues must be addressed when attempting to use EEG systems within the MRI system. The introduction of the EEG equipment into the pulsed RF fields created by the MRI scanner can also present a safety hazard, especially at high static B0 fields, because of specific absorption rate (“SAR”) considerations. EEG leads may act as antennas, increasing the patient's exposure to the RF fields. The use of metallic electrodes and leads may cause an undesirable increase in local and whole-head SAR values, reflected in the heating of the patient's tissue. Such heating may result in bodily injury to the patient, including burns, electric shock, etc. Further still, the noise created by motion can degrade both the fMRI and the EEG data, but in distinct ways.

Similar challenges exist when EEG systems are used with other imaging modalities. For example, when EEG is used with computed tomography (CT) or other x-ray-based imaging, the EEG system and its inherent opacity can attenuate the x-rays and interfere with the ultimate images. Similar problems exist with other imaging modalities, where the size of the EEG system may cause positrons to be absorbed or scattered and interfere with positron emission tomography (PET) imaging.

Wider clinical and research use of EEG is further limited by its size and complexity in other ways. For example, EEG can be difficult to use with children, particularly very-young children, because the size of the child's head may not be able to accommodate the substantial space required to engage with an array of EEG leads, such as 128 leads. Thus, in some attempts to perform EEG on children, clinicians are left to manually adhere as many individual EEG electrodes to the child as possible. Of course, even beyond the inefficiency, this often does not produce consistent or readily reproducible data.

Thus, there is a need for EEG systems that have fewer limitations, including limitations on patient compatibility and/or compatibility with imaging systems.

### SUMMARY

The present disclosure overcomes the aforementioned drawbacks by providing systems for EEG and methods of manufacturing those systems that offer small components and more flexibility than traditional EEG systems. For example, the EEG system may be manufactured through a combination of thin-film and polymer thick film technologies to create individual EEG lead systems that can be coupled with a support structure, such as a cap, that can be sized for a child, even while including an extensive number of individual leads, such as 128 leads or more. In this way, a conductive trace may be printed on a base layer or substrate with a height extending above the base layer of 50 nm or less, including 30 nm. An EEG electrode may be formed over the conductive trace using a polymer thick film arranged over the conductive trace at one end of the base layer.

In accordance with one aspect of the disclosure, a system is provided for transmitting electrical signals to or from a subject's head. The system includes a support structure configured for placement on a head of a human subject and at least one lead system coupled to the support structure. At least one lead assembly includes a base layer having a top surface, a bottom surface extending from a proximal end to a distal end, an electrical connector attached to a proximal end of the base layer, and a conductive circuit. The conductive circuit includes a conductive trace printed as a thin film along the top surface of the base layer and extending between and electrically connecting the electrode and the electrical connector. An electrode formed printed on top of the conductive trace at the distal end of the base layer. A height of conductive trace as measured from the top surface of the base layer is less than about 50 nm.

In accordance with one aspect of the disclosure, a system is provided for transmitting electrical signals to or from a subject's head. The system includes a base layer with a top surface, a bottom surface, a proximal end, a distal end, an electrical connector attached to a proximal end of the base layer, and a conductive circuit. The conductive circuit includes an electrode supported along the top surface of the base layer at the distal end of the base layer and a conductive trace supported along the top surface of the base layer, the conductive trace extending between and electrically connecting the electrode and the electrical connector. The conductive trace is formed from a conductive, non-ferrous material deposited along the upper surface of the base layer using a thin-film deposition process. The electrode is formed as a polymer thick film printed on the conductive trace at the distal end of the base layer.

In accordance with one aspect of the disclosure, a method is provided for forming a system for transmitting electrical signals to or from a subject's head. The method includes providing a base layer and depositing a layer of conductive, non-ferrous material along an upper surface of the base layer using a thin-film deposition technique. The method also includes patterning the deposited non-ferrous material into a conductive trace that extends between a proximal end and a distal end of the base layer and electrically coupling a thick film forming an electrode relative to a distal end of the conductive trace.

The foregoing and other aspects and advantages of the invention will appear in the following description. In the description, reference is made to the accompanying drawings, which form a part hereof, and in which a preferred embodiment of the invention is shown by way of illustration. Such embodiment does not necessarily represent the full scope of the invention, however, and reference is made therefore to the claims and herein for interpreting the scope of the invention.

### BRIEF DESCRIPTION OF THE DRAWINGS

The foregoing and other aspects of the present disclosure will be apparent upon consideration of the following detailed description, taken in conjunction with the accompanying drawings, which reference characters refer to like parts throughout.

FIG. 1 is a block diagram of an exemplary system for recording EEG signals within an MRI environment configured in accordance with the present disclosure.

FIG. 2 is an illustration of an EEG system according to one example aspect of the present disclosure.

FIG. 3 is a perspective view of a mounting system of a carrier assembly according to one example aspect of the present disclosure.

FIG. 4 is a top view of one lead system according to one example aspect of the present disclosure.

FIG. 5 is an exploded top view of the lead assembly of FIG. 4 according to one example aspect of the present disclosure.

FIG. 6 is a schematic illustration of a one example circuit of the lead system of FIG. 4.

FIG. 7A is a cross-sectional view of the electrode of the lead system of FIG. 4 taken along line 7A-7A according to one example aspect of the present disclosure;

FIG. 7B is a cross-sectional view of the connector of the lead system of FIG. 4 taken along line 7B-7B according to one example aspect of the present disclosure;

FIG. 8 is a cross-sectional view of the lead assembly of FIG. 4 taken along line 8-8 according to one example aspect of the present disclosure;

FIG. 9

FIG. 10A shows point-specific absorption rate (pSAR) results obtained during MRI RF safety testing of a voxel model with no EEG leads present.

FIG. 10B shows point-specific absorption rate (pSAR) results obtained during MRI RF safety testing of a voxel model with a carrier assembly according to one example aspect of the present disclosure.

FIG. 10C shows point-specific absorption rate (pSAR) results obtained during MRI RF safety testing of a voxel model with a commercially available EEG cap, including EEG leads formed with copper traces.

FIG. 11A is a reference map illustrating the locations of lead assemblies and fiber optic probes during testing of RF-induced heating of a carrier assembly according to one example aspect of the present disclosure.

FIG. 11B is a plot of temperatures measured during testing of RF-induced heating of a carrier assembly incorporating lead assemblies according to one example aspect of the present disclosure.

### DETAILED DESCRIPTION

As will be described, the present disclosure provides systems and methods for Electroencephalography (EEG). More particularly, the present disclosure provides systems and methods for EEG that are inexpensive, noninvasive, and overcome current cross-modal safety and artifact issues that severely limit the effectiveness of simultaneous high-definition (HD) EEG and magnetic resonance imaging (MRI) or computed tomography (CT) imaging. Thus, the systems and methods enable the anatomic images with high spatial resolution, such as MRI or CT imaging, to be coupled with the high temporal resolution of HD-EEG. The EEG systems and methods provided herein can be used to produce lightweight and small systems, such as can even be used with small children. Thus, the systems and methods provided herein allow for the study of brain function in healthy neonates in natural settings and the understanding of different neonatal neural pathologies, such as epilepsy. In addition to MRI/CT, the systems and methods provided herein can produce extended EEG lead assemblies using non-ferromagnetic components that allow the EEG system to be concurrently used with other imaging technologies, including positron emission tomography (PET), Single Photon Emission Computed Tomography (SPECT), transcranial magnetic stimulation (TMS), and magnetoencephalography (MEG).

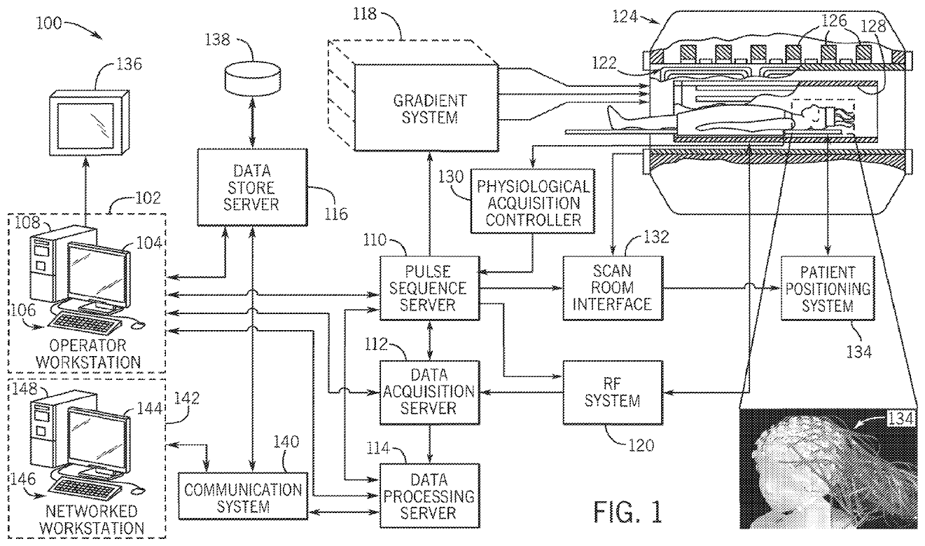

Referring now to FIG. 1, the systems and methods provided herein may be utilized with a magnetic resonance imaging (MRI) system 100, which may be configured, programmed, or otherwise utilized in accordance with the present disclosure, such as in coordination with an EEG system 101. Though FIG. 1 illustrates an MRI system, it represents a general imaging modality that can be used with the systems and methods of the present disclosure. Thus, the MRI system 100 of FIG. 1 could be substituted for any of a variety of other imaging modalities, such as CT, PET, SPECT, or the like, and/or can be used with TMS or MEG. Thus, the MRI system 100 is illustrated as just one non-limiting example of an imaging modality. However, MRI systems 100 are generally one of the more difficult modalities to utilize with additional sensor systems, such as the systems that will be described, due to the limitation against ferromagnetic components and specific absorption rate (SAR)/heating considerations. However, as will be shown herein, the systems and methods of the present disclosure are able to manage and overcome the SAR and heating limitations common to many EEG systems. Thus, the MRI system 100 is provided as an example of an imaging system that inherently presents substantial co-use limitations on traditional EEG systems. Furthermore, as will be described, in one non-limiting example, the systems and methods of the present disclosure, in one configuration, may include additional motion tracking capabilities that can be coordinated with an MRI system, such as the MRI system 100 of FIG. 1 to track and compensate or adjust for motion.

The MRI system 100 includes an operator workstation 102, which will typically include a display 104, one or more input devices 106 (such as a keyboard and mouse or the like), and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to multiple servers, including a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include both proprietary or dedicated networks, as well as open networks, such as the internet.

The pulse sequence server 110 functions in response to instructions downloaded from the operator workstation 102 to operate a gradient system 118 and a radiofrequency (RF) system 120. Gradient waveforms to perform the prescribed scan are produced and applied to the gradient system 118, which excites gradient coils in an assembly 122 to produce the magnetic field gradients Gₓ, G_{y}, and G_{z} used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128 or a separate local coil such as, e.g., an optional surface coil configured to be positioned against an intended imaging target of a patient in order to perform the prescribed magnetic resonance pulse sequence. Responsive magnetic resonance signals detected by the RF coil 128, or a separate local coil (e.g., an optional surface coil), are received by the RF system 120, where they are amplified, demodulated, filtered, and digitized under the direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or one or more local coils (e.g., optional surface coils) or coil arrays.

The RF system 120 also includes one or more RF receiver channels. Each RF receiver channel includes an RF preamplifier that amplifies the magnetic resonance signal received by the coil 128 (e.g., an optional surface coil) to which it is connected and a detector that detects and digitizes the I and Q quadrature components of the received magnetic resonance signal. The magnitude of the received magnetic resonance signal may, therefore, be determined at any sampled point by the square root of the sum of the squares of the I and Q components:

$$M = \sqrt{{I}^{2} + {Q}^{2}} ; \qquad Eqn . 1$$

and the phase of the received magnetic resonance signal may also be determined according to the following relationship:

$$φ = {tan}^{- 1} ( \frac{Q}{I} ) . \qquad Eqn . 2$$

The pulse sequence server 110 also optionally receives patient data from a physiological acquisition controller 130. By way of example, the physiological acquisition controller 130 may receive signals from a number of different sensors connected to the patient, such as electrocardiographic (ECG) signals from electrodes or respiratory signals from respiratory bellows or other respiratory monitoring devices. Such signals are typically used by the pulse sequence server 110 to synchronize, or “gate,” the performance of the scan with the subject's heartbeat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage such that no data are lost by data overrun. In some scans, the data acquisition server 112 does little more than passing the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data are acquired and used to calibrate the pulse sequence performed by the pulse sequence server 110. As another example, navigator signals may be acquired and used to adjust the operating parameters of the RF system 120 or the gradient system 118 or to control the view order in which k-space is sampled.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction techniques, such as iterative or backprojection reconstruction techniques; applying filters to raw k-space data or reconstructed images; generating functional magnetic resonance images; calculating motion or flow images; and so on.

Images reconstructed by the data processing server 114 are conveyed back to the operator workstation 102. Images may be output to operator display 112 or a display 136 located near the magnet assembly 124 for use by the attending clinician. Batch mode images or selected real-time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. Images reconstructed by the data processing server 114 are conveyed back to the operator workstation 102. Images may be output to operator display 112 or a display 136 located near the magnet assembly 124 for use by the attending clinician. Batch mode images or selected real-time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

As will be described in further detail below, the MRI system 100 and the EEG system 101 may be configured to operate together in coordination. Thus, as will be described, the EEG system 101 may include a plurality of EEG leads configured to be positioned about a head of a patient arranged in the bore of the MRI system 100 to acquire EEG data while acquiring MRI data. Thus, the EEG leads of the EEG system 101 are MRI compatible. Furthermore, the EEG system may be integrated with or may be coupled to a sensor system to form a sensor assembly capable of tracking patient position or motion in addition to acquiring EEG and/or MRI data. By tracking the position or motion of the patient during MRI and EEG data acquisition, the systems and methods of the present disclosure are able to track physiological signals, such as brain signals, from the patient with high spatial and temporal resolution. Thus, as will be described, the EEG system is capable of recording the EEG signals within an MRI environment.

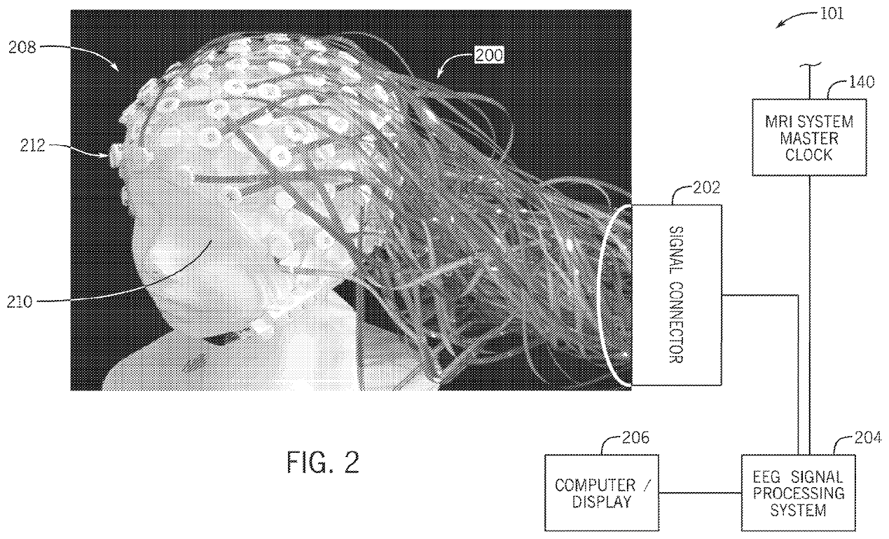

Referring to FIG. 2, the EEG system 101 is illustrated in further detail. In particular, the EEG system 101 can include a lead assembly 200. The lead assembly 200 extends to a signal connector 202. The signal connector 202 may include any number of different connectors (e.g., a NICOMATIC connector) via which patient electrical signals obtained by the lead assembly 200 may be transferred to an EEG signal processing system 204. The EEG signal processing system 204 may include amplifiers, transmitters, receivers, A/D converters, filters, and any other circuitry or component that may be used to process the acquired signals (e.g., EEG signals) for use and display by an EEG computer and display system 206.

It should be understood by one skilled in the art that the EEG signal processing system 204 or portions of the EEG signal processing system 204 may be located outside of the MRI system 100 of FIG. 1 or the MR-shielded room. For example, the amplifiers and transmitters may be located inside the MRI-shielded room and connected via optical fiber to the receivers located outside the MRI-shielded room. It should also be understood by one skilled in the art that additional circuitry and components may be included in or associated with the EEG system 101, such as electrical stimulators and matrix switches for use in EIS and EIT recordings and adaptive filters for filtering out the noise. It should further be understood by one skilled in the art that the EEG computer/display system 206 may execute procedures for processing the EEG signals acquired within the MRI system of FIG. 1 or within the MR-shielded room.

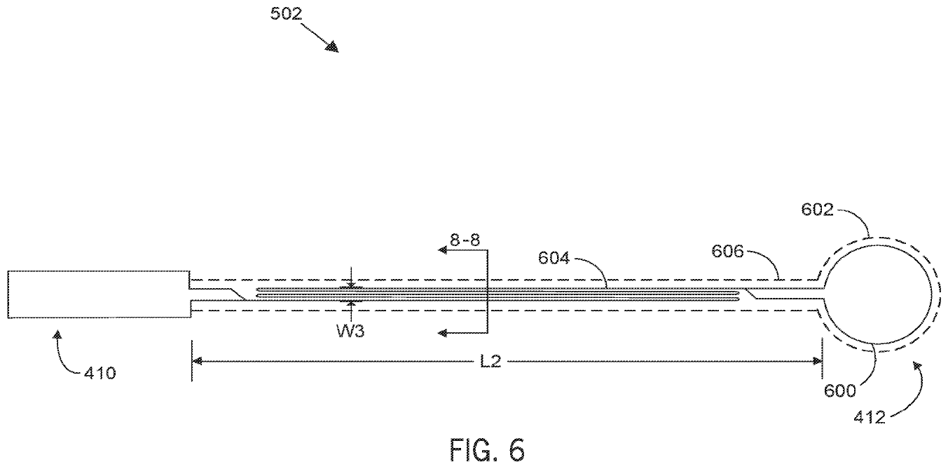

As will be described, in some non-limiting configurations, the EEG system 101 may include individual EEG lead systems of the lead assembly 200 that include both an EEG electrode and a coil or loop configured to acquire position or movement data. In this case, the EEG signal processing system 204 and/or computer 206 may be connected to an MRI system clock 140. That is, the MRI system 100 includes a master clock 140 that connects to the various parts of the MRI system 100 of FIG. 1. For example, the master clock 140 may connect to components of FIG. 1, such as the RF system 120 and the gradient system 118, as just one example. In this way, though the MRI system 100 operates a clock frequency that is generally appreciably greater than the EEG system 101, such as 10 MHz versus less than 50 Hz, the master clock 140 provides a resource through which to synchronize the MRI data with the data from the EEG system 101. In this way, the noise can be systematically sampled so that, at each repetition time (TR) of the MR pulse sequence, the noise is sampled in the same fashion and appears to be the same. That is, as will be described, the EEG system 101 can include a combined EEG electrode and motion coil, such as will be illustrated in FIG. 6, that, together, allow motion or position information to be identified

As will be described in detail, the EEG lead assembly 200 may include a carrier assembly 208 that may include a support structure 210 that is engaged by a plurality of mounting systems 212. The support structure 210 is desired to position a plurality of EEG sensors about a patient's head via the mounting systems 212 that engage the support structure 210. As will be described, EEG lead assembly, thus, provides a system that is designed to acquire patient signals from multiple patient sites during an EEG procedure (e.g., during a combined EEG/MRI procedure).

The support structure 210, as illustrated, can be configured to be attached to (e.g., worn by) a patient during an EEG procedure. The support structure 210 can be designed to be invisible to MRI. For example, the support structure 210 may be constructed from silicone or other MR-invisible/MR-compatible material. The configuration of the support structure 210 is adapted to the patient's particular anatomy from which signals are to be obtained. For example, as illustrated by FIG. 2, the support structure 210 can be configured as a cap fitted to a patient's head.

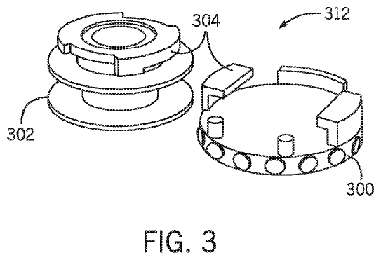

As shown in FIG. 2 and FIG. 3, according to one configuration, the mounting system 212 includes a plurality of pairs of sensor mounts 300 and the corresponding locking mounts 302. Each sensor mount 300 is supported relative to an exterior surface of the support structure 210 and is configured to receive and position a distal, patient signal-acquiring end of a lead assembly 200 against the patient when the support structure 210 is positioned on the patient. A corresponding locking mount 302 mates with the sensor mount 300 to fixedly secure an EEG sensor (not shown yet) of the lead assembly 200 in the mounting system 212 relative to the support structure 210. For example, a set of inter-engaging locking keys 304 formed on each of the sensor mount 300 and locking mount 302 may allow the locking mount 302 and the sensor mount 300 to lock the distal end of the lead assembly 200 received therebetween, and thus secure an EEG sensor relative to the support structure 210.

The number, spacing, and arrangement of the sensor mounts 300 about the support structure 210 may be varied as desired based on the desired patient signals to be acquired. As described in more detail below, the small and lightweight construction of the lead assemblies 200, as well as the minimal amounts of conductive material contained by each lead assembly 200, allows for an increased amount of lead assemblies 200 to be incorporated into the carrier assembly 208 without adversely impacting patient safety (e.g., without increasing SAR) and without impacting signal quality. Accordingly, in various configurations, the support structure 210 may be designed to receive a high density (e.g., more than 128, and in some embodiments, 256 or more) of the mounting systems 212. As will be appreciated, not all configurations dictate that all potential locations for receiving a mounting systems 212 on the support structure 210 need to be utilized during any given situation.

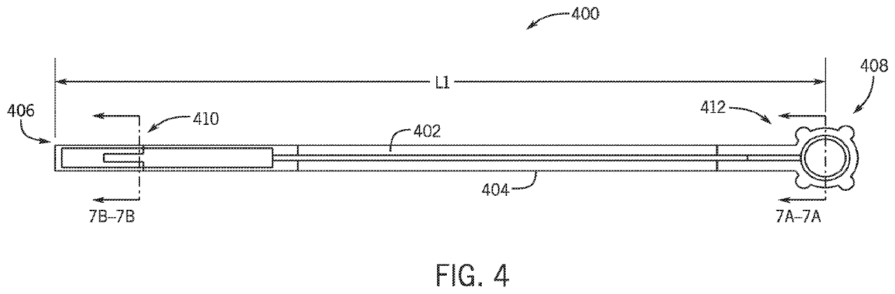

Referring to FIG. 4, an individual lead system 400 of the lead assembly 200 of FIG. 2 is illustrated. As illustrated in FIG. 4, the lead system 400 includes a first, top surface 402, and a second, bottom surface 404 that each extend between a first, proximal end 406, and a second, distal end 408 to define a length L1. The proximal end 406 of the lead system 400 includes a connector 410 that is designed to connect to the signal connector 202 of FIG. 2. The distal end 408 of the lead system 400, as will be described, includes at least one sensor 412 and may include an electrode and an additional sensor, such as a coil or loop. As will be described, the distal end 408 is configured to be secured relative to a portion of a patient (e.g., the head of a patient) from which it is desired to obtain electrical signals. That is, the distal end 408 is configured to be coupled with the sensor mount 300 of FIG. 3. The distal end 408 of the lead system 400 is configured to be received by the sensor mount 300 of the mounting system 212 and secured against movement relative to the support structure 210 of FIG. 2 via an inter-engagement between the locking key 304 of the sensor mount 300 (within which the distal end 408 of the lead system 400 is received) and the locking mount 302.

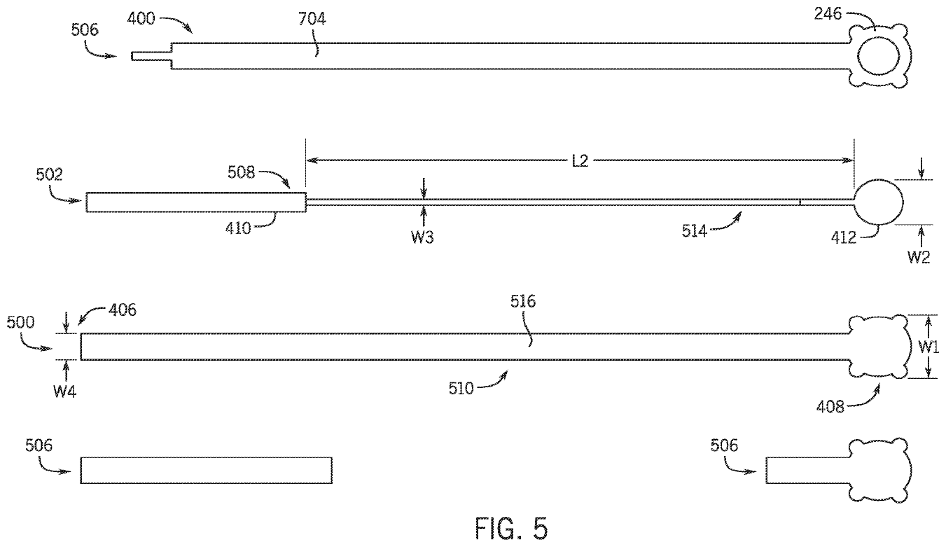

Referring now to FIG. 5, an exploded view of the lead system 400 of FIG. 4 is provided. As shown in FIG. 5, the lead system 400 may be formed from a base layer 500, a conductive circuit 502, and a cover layer 504. According to some configurations, the lead system 400 can also include one or more optional structural elements 506, for example, such as may be configured to provide additional rigidity to the lead system 400 when assembled.

The conductive circuit 502 provides an electrical pathway to obtain and transmit electrical signals from a patient to an EEG signal processing system, as described above. The conductive circuit 502 is arranged on the base layer 500. The base layer 500 can be formed of one or more layers made of one or more non-conductive materials. In general, the one or more materials forming the base layer 500 are selected to allow the base layer 500 to withstand the various conditions (e.g., temperatures, chemicals, etc.) to which the base layer 500 may be subject during the trace-forming process (described in detail below), and to enhance adhesion of the base layer 500 to other components of the lead system 400, such as a conductive trace 508 of the conductive circuit 502 and the cover layer 506, and the like. Non-limiting examples of materials that may be used to form the base layer 500 include polyimide, liquid crystal polymer, or other polymers having high heat and/or chemical stability.

As shown in FIG. 5, the base layer 500 includes a central portion 510 that extends between the first proximal end 406 and the second distal end 408. As described in more detail below, a first, upper surface 516 of the base layer 500 supports the conductive circuit 502. The proximal end 406 of the base layer 500 provides an attachment surface via which the conductive circuit 502 of the lead system 400 may be attached (e.g., via crimping) to the electrical connector 410 of FIG. 4.

A length of the base layer 500 may be dimensioned based on the use of the lead system 400. For example, according to various configurations, the length of the base layer 500 maybe 50 cm or longer, so as to allow the lead system 400 to be used to measure EEG signals during various imaging procedures (e.g., MRI, computed tomography (CT), positron emission tomography (PET), single-photon emission computed tomography (SPECT), transcranial magnetic stimulation (TMS), magnetoencephalography (MEG), etc.).

The base layer 500 is dimensioned to provide the upper surface 516 along which to support the components of the conductive circuit 502. For example, a width W1 of the distal end 408 of the base layer 500 will correspond to a width that accommodates the widest portion of the conductive circuit 502, which is the sensor 412 located at the distal end 408. According to some configurations, the W1 of the distal end 408 of the base layer 500 may be about 11 mm.

As described in more detail below, thin-film deposition and trace-patterning processes can form the lead system 400 to advantageously allow the conductive trace 508 of the conductive circuit 502 to be defined by a narrow width and a compact trace pattern. Accordingly, as described in more detail below, in various configurations, and overall width W3 of the conductive trace 508 is generally much narrower than the width W2. Thus, a width W4 of a central portion 514 of the base layer 500 may be constructed (e.g., via laser cutting of the base layer 500) to be narrower than the width W1 of the distal end 408 of the base layer 500. Minimizing the width W4 of the base layer 500 along the central portion 514 and the proximal end 406 may advantageously decrease the overall weight and size of the lead system 400. According to some configurations, a width W4 of the central portion 514 and the proximal end 406 of the base layer 500 may be less than about 5 mm, and in some configurations, as narrow as about 0.12 mm.

At the distal end, 408, sensor 412 is electrically connected to the conductive trace 508. The sensor 412 is configured to be engaged by the mounting system 212 of FIG. 2 to engage the patient (e.g., proximate to the scalp of a patient) without the need for adhesives or the like. As will be described, according to some configurations, a width W2 (i.e., a diameter) of the sensor 412 may be between about 5 mm and about 8 mm.

As shown in FIG. 6, circuit 502 is shown in detail. The sensor 412 is shown in detail as including one or more components. The sensor 412 includes an electrode 600. In one non-limiting configuration, as will be described, the electrode 600 may be encircled by an optional coil or loop 602. If included, the coil/loop 602 can be coupled to a respective conductive trace 606. If both conductive traces 604 and 606 are included, they may be electrically isolated. For example, the conductive traces 604 and 606 may be arranged on opposite sides of the base layer 500 of FIG. 5. The conductive traces 604 and 606 may reflect a variety of different trace patterns (e.g., various widths, overall effective lengths, spacings in between adjacent portions of the conductive traces, etc.). In the non-limiting example of FIG. 6, a trace pattern of the conductive trace 604 connected to the electrode 600 may have a non-linear shape. In the non-limiting example, the non-linear shape is a serpentine shape of width W3. In this example, W3 may indicate a physical width of less than about 1 mm, preferably less than about 100 μm, more preferably less than about 25 μm, and in some embodiments, about 20 μm. Alternatively, one or more of the conductive traces 604 and 606 may have another shape. For example, in the illustrated non-limiting configuration, a trace pattern of the conductive trace 604 extending from the electrode 600 forms a single straight line that extends across a length of the other conductive trace 606 to extend a length L2 or further.

In some embodiments, the use of a non-linear trace pattern for one conductive trace 606 may be selected to achieve an overall effective length that is capable of attaining the desired impedance profile tailored to specific MRI field strengths or applications with specific pulse sequences with differing RF characteristics. As another example, such as is the case of electrical impedance spectroscopy (“EIS”) or electrical tomography (“EIT”) performed using the electrode 600, the trace pattern of the conductive trace 604 may be selected based on the desired impedance profile determined using specialized acquisition system software. In yet other configurations, the trace pattern may alternatively or additionally be selected to attain a variable resistance along the length of the conductive trace 604, 606 that is tailored to a specific desired frequency response attenuation for a particular recording application. Further trace pattern customizations of the conductive trace 604 and 606 may be implemented to additionally or alternatively optimize the performance of the overall lead assembly for specific MRI manufacturers and models, specific bore configurations, head coils, and EEG recording systems.

The electrode 600 (and/or other conductive components of the conductive circuit 502) may be deposited (e.g., through the use of thick-film technology, such as a polymer thick film (PTF)) onto the upper surface 516 of the base layer 500 of FIG. 5. Thick-film technology is generally used to produce electronic devices or components, such as surface mount devices, hybrid integrated circuits, heating elements, and the like. Thick-film technology creates conductors by stenciling or screen printing. Generally, thick-film, particularly PTF, yields electronics that are fractions of a millimeter in thickness (e.g., 0.01 mm to 1 mm).

The electrode 600 may be designed in a variety of configurations. Even when created using thick film, in one non-limiting example, the electrode 600 may form a generally thin pad (e.g., a circular, semi-circular, or otherwise shaped flat disc or other structure). The electrode 600 may be constructed from any conductive biocompatible materials capable of providing conductivity at levels suitable for performing the EEG or other electrical measurements (e.g., silver, silver chloride, etc.). In some non-limiting examples, the electrode 600 or traces 604 extending therefrom may be formed using conductive ink. In such configurations, the conductive ink(s) used as the electrode-forming material may include one or more conductive materials such as metals (e.g., copper or silver) and/or metal ions (e.g., silver chloride), filler-impregnated polymers (e.g., polymers mixed with conductive fillers such as graphene, conductive nanotubes, metal particles), or any ink having a conductive material capable of providing conductivity at levels suitable for performing the EEG or other electrical measurements. In various embodiments, inks having a mixture of silver and silver chloride conductive inks may be desirable for the electrode-forming material as they generate conductive pathways without the use of sintering or other high-temperature operations and thus may enable the use of a broader range of high temperature-sensitive materials for the base layer 500 of FIG. 5

The electrode 600 is coupled to the conductive trace 604. The traces may be formed using thin-film (TF) technology. In this way, manufacturing of conductive leads 604 may use TF technology, which is combined with a polymer thick film (PTF). That is, PTF may be paired with TF to achieve unprecedented trace density in an EEG assembly arranged onto a carrier assembly 208 that allows the routing of many traces in an extremely small space. Depositing thin film onto polymer substrates 500 is extremely complicated, given the extreme sensitivity of polymers to heat. Generally, thin-film technology applies materials ranging from fractions of nanometers to nanometers in thickness. Thus, thin firm technology generally operates in an order of magnitude less in thickness than thick film technology. The transition from thick to thin-film requires many complexities to be managed and overcome.

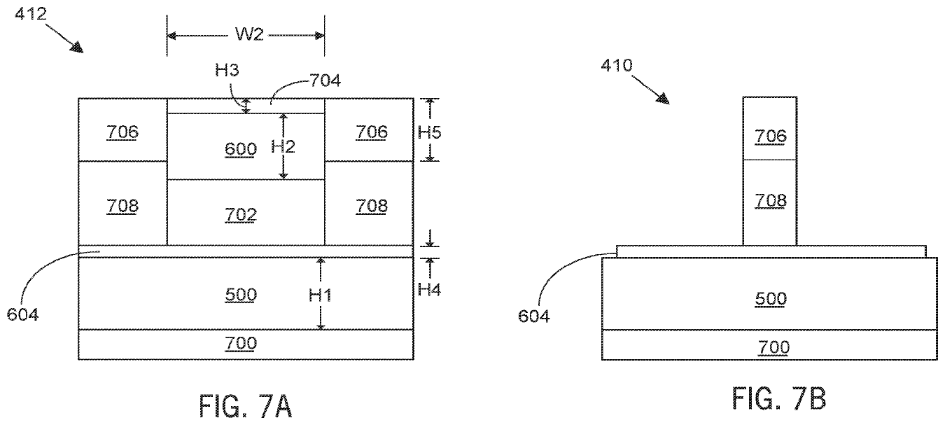

For example, as will be described in further detail with respect to FIGS. 7A and 7B, the base layer 500 or substrates may include polyimide and liquid crystal polymer, which can accept a very thin metallization (~10 nm) for 75 μm thick of Aluminum to form the trace 604 and/or electrode 600. Aluminum coatings, such as anodize or chem-film, are biocompatible and Restriction of Hazardous Substances (RoHS) compliant. Aluminum is one of the less dense metals, so it can be used in CT or X-rays imaging environments while presenting substantially reduced artifacts in the images. The use of thin-films to form the traces 604 is a potentially inexpensive solution compared to other materials that have been attempted, such as those made completely out of polymer thick film and containing an expensive amount of silver flakes. Plastic films coated with aluminum can be used where the metallization transforms the plastic film into a film impermeable to moisture and bacteria. The aluminum layer is a biocompatible barrier. The thin film traces, as described herein, are very narrow, as narrow as human hair. This dimension comes with at least two advantages compared to traditional systems. First, many traces can be fit in a small space, which is ideal for many patients, including pediatric patients (lightweight, fewer cables reduce the risk of tangling and future expandability). Second, the small dimensions and materials facilitate MRI and CT compatibility. Also, as described herein, the overall lead systems can include long traces, which is traditionally challenging. However, the systems and method provided herein are able to form traces in thin-film that is longer than, as just one example, 20 inches because photolithography using dry photo resistive film, and aluminum etching, overcomes the failings of traditional attempts. The PTF can be printed on top of the TF. For example, PTF printing on top of the TF trace 604 can be used to create the electrodes 600 (which can be printed in Ag/AgCl) or create a dielectric for encapsulation of the traces. In the latter application, the PTF printing can use TPU filaments that complete the trace manufacturing, which gives electrical insulation, scratch protection, and chemical insulation. In one non-limiting example, a vacuum-coating may be used that provides an adhesion promoter to ensure substrate/cap lattice structure adhesion. Finally, a silicone, Parylene, or TPU coating can be applied. One non-limiting example of the system will be described with respect to FIGS. 7A and 7B.

Referring to FIGS. 7A and 7B, a cross-section of non-limiting examples of the sensor 412 and the connector 410, respectively, of FIG. 4, are provided. The sensor 412 and connector 410 may be formed by a one or more subcomponents

As previously described, sensor 412 and connector 410 are coupled to the base layer 500. The connector 410 may be formed as traces/electrodes of FIG. 7A are crimped to a flex circuit configured to interface with a connector for connection to the EEG amplifier or other components of an EEG system. The exposed traces and crimped interconnect can be waterproofed, for example, using Kapton and 3M Extreme Sealing 4411 tapes.

The base layer 500 may have a thickness (i.e., height H1) selected to allow the base layer 500 to provide sufficient structural support to the sensor 412. However, according to various configurations, it may be advantageous to minimize the thickness (i.e., height H1) of the base layer 500 as much as possible (i.e., without sacrificing the structural support that the base layer 500 is intended to provide). Notably, minimizing the thickness of the base layer 500 may increase the flexibility of the lead system 400, thereby allowing the lead system 400 to more easily adapt to the contours of the anatomy of a patient to which the lead assembly 200 of FIG. 2 is applied (e.g., the head of a patient). According to one non-limiting example embodiment, the base layer 500 is formed from KAPTON HN, and has a height H1 of about 75 μm.

Depending on the desired flexibility and the specific configurations, one or more stiffening carriers 700 may be joined with the base layer 500. The stiffening elements 700 are formed from a material having a stiffness greater than that of the base layer 700. The stiffening elements 700 are configured to be secured to a second, lower surface of the base layer 50 (e.g., via an adhesive) to provide structural reinforcement to portions of the base layer 500 that may be susceptible to stress and/or strain.

The conductive trace 604 may extend along the base layer 500. The conductive trace 604 may take a variety of different forms. In one non-limiting example, the conductive trace 604 has a resistance that may be between about 10 kΩ and about 15 kΩ. The trace-forming process includes a thin-film deposition step. During the thin-film deposition step, a very thin layer (i.e., a layer having a thickness of less than about 100 nm, preferably less than about 30 nm, and in some embodiments a thickness of about 10 nm) of trace-forming material is deposited along the base layer 500. The height of the trace-forming material deposited during the thin-film deposition step corresponds to a height H4 of the formed conductive trace 604. The conductive trace 604 can have a very minimal height H4. Non-limiting examples of thin-film fabrication methods via which the trace-forming material may be deposited include metallization, chemical or physical vapor deposition, plating, chemical solution deposition, spin coating, chemical vapor deposition (“CVD”), sputtering, or other suitable processes that allow for the deposition of the trace-forming material with a thickness of less than about 1 μm, and preferably less than about 100 nm, and more preferably less than about 50 nm.

The trace-forming process can also include a trace-patterning step. According to some configurations, during the trace-patterning step, portions of the trace-forming material deposited on the base layer 500 during the thin-film deposition step are selectively removed to create a conductive trace 604 defined by a trace design having the desired width, length, and trace pattern. The trace patterning step allows for the precise and controlled removal of select portions of the deposited trace-forming material, thereby allowing for the patterning of high tolerance trace designs. Non-limiting examples of methods that may be used to remove the deposited trace-forming material selectively include lithography, etching, trimming, lift-off, or other suitable processes that allow for the patterning of a conductive trace 604 with a width of less than about 1 mm, preferably less than about 100 μm, more preferably less than about 25 μm, and in some embodiments about 20 μm.

Alternatively, in some configurations, the trace-patterning step may involve the application of a mask or stencil having a desired trace pattern onto the base layer 500. Once the desired trace pattern has been outlined, the trace-forming material is deposited during a thin-film deposition step (and mask/stencil applied along a portion thereof), following which the mask/stencil is optionally subsequently removed.

As will be appreciated, the trace-forming process may include steps in addition to the thin-film deposition step and the trace-patterning step described above. For example, prior to the thin-film deposition step, the trace-forming process may include steps in which the base layer 500 and/or the trace-forming material undergoes treatments (e.g., surface patterning treatments, the addition of chemical modifiers, etc.) to enhance the bonding between the trace-forming material to the base layer 500 (i.e., to minimize the risk of delamination of the conductive trace 220 from the base layer 500). Following the thin-film deposition step, the trace-forming process may optionally include one or more steps (e.g., curing, polymerization, solvent evaporation, vacuum-coating with an adhesion promotor, etc.) to enhance the bond formed between the base layer 500 and deposited trace-forming material.

According to one example embodiment, the thin-film deposition step may include vacuum coating the base layer 500 with a thin (e.g., 30 nm) layer of aluminum. The aluminum may include anodized or chem-film aluminum. A dry photoresist mask is applied to the aluminum-coated base layer 500. Photolithography is used to selectively etch a desired trace pattern from the deposited aluminum to form the conductive trace 604. Following the etching procedure, the dry photoresist mask may be removed. Alternatively, in some embodiments, the dry photoresist mask may be selected from a biocompatible material and may be left on the upper surfaces of the remaining portions of the aluminum (i.e., the dry photoresist mask is not removed following the etching procedure) that define the conductive trace 604 following the etching procedure. In such embodiments, the remaining dry photoresist mask may act as a protective layer for the conductive trace 604.

As described above, the conductive trace 604 is electrically coupled with the electrode 600. For example, a conductive adhesive 702 may be used to couple the conductive trace 604 to the electrode 600. In one non-limiting example, the conductive adhesive 702 may be a carbon-based conductive glue. Regardless of the particular adhesive, the present disclosure notes that the adhesive 802 is designed to provide a non-destructive chemical bond while maintaining conductivity between the electrode 600 and the conductive trace 604. Notably, not all adhesives achieve these ends. For example, some Ag-based inks can destroy a thin aluminum trace that may be used to form the conductive trace 604.

A silver foil may be arranged as a pad forming the electrode 600. Additionally or alternatively, the electrode 600 may optionally be provided with one or more suitable coating layers 704 (e.g., a silver chloride coating, a coating of gold, etc.). In some configurations, one or more of the coating layers 704 may be formed onto an upper surface of the electrode 600 once the electrode 600 has been secured onto/formed along the base layer 500. Alternatively, or additionally, one or more of the coating layers 704 may be deposited onto the upper surface of a discretely provided electrode 600 prior to the integration of the discretely provided electrode 600. For example, according to some configurations, the coating layer 704 may include a thin layer of silver chloride (i.e., having a height of about 100 nm or less) that is coated (e.g., via a chemical process) along an upper surface of an electrode 600 formed from a thin (e.g., having a height H2 of about 100 μm or less, and preferably a height of about 25 μm or less) sheet (e.g., a foil) of silver. In some configurations, the electrode 600 may additionally, or alternatively, include a coating layer 702 that includes a thin layer (e.g., having a height H3 of about 300 nm or less) of gold that is vacuum coated onto the electrode 600 (e.g., via electron-beam vapor deposition).

A cover layer 706 may be included to provide different properties and/or forms of protection. For example, the cover layer 706 may provide electrical insulation, waterproofing, chemical insulation, and protection against physical damage (e.g., scratching, delamination from the base layer 500, etc.). The cover layer 706 may include one or more layers formed from similar or different materials that may be applied.

The cover layer 706 may include a discretely provided protective sheet (e.g., a silicone sheet, a Pyralux sheet, a Parylene sheet) that is attached (e.g., via an adhesive 708 such as epoxy of LOCTITE, via vacuum bonding, thermal bonding, etc.) along an upper surface of the conductive circuit 604. Alternatively, or additionally, the cover layer 706 may be formed from one or more protective layer-forming materials that are deposited (e.g., via a coating, printing, spraying, thick-film process, etc.) to form the cover layer 706 in situ. According to one non-limiting example, a height H5 of the cover layer 706 (formed, e.g., from a Pyralux protective sheet) may be about 25 μm.

In general, the cover layer 706 may extend along less than an entirety of the upper surface of the sensor 412, as illustrated. For example, as shown in FIG. 5, the cover layer 706 may form a window or passage at the distal end 408, that is of size W2 illustrated in FIG. 6A, such that the electrode 600 is exposed to the patient (e.g., via the cover layer 704). A diameter W2 of the window is generally sized to correspond to a width W2 of the electrode 600 (i.e., a diameter of the window is equal to or is slightly less than the width W2 of the electrode 600).

Thus, in one non-limiting example, six layers may be used to form sensor 412. In this non-limiting example, the six layers may include (1) a polyimide substrate or base layer, (2) a polyimide stiffener, which may be arranged on one or both sides of the substrate, (3) an aluminum metallization, (4) a carbon-based glue, (5) a silver electrode pad, and (6) an AgCl layer to improve the recording sensitivity of the electrode pad for EEG recordings.

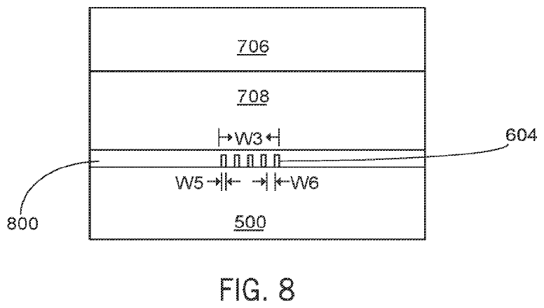

As illustrated in FIG. 8, between the sensor 412 shown in the cross-section in FIG. 7A and the connector are shown in the cross-section in FIG. 7B, the conductive trace extends. As described with respect to FIG. 6, the conductive trace 604 may be linear or non-linear. One non-limiting example of a cross-section of the conductive trace 604 in a non-linear configuration is illustrated in FIG. 8. In some configurations, in addition to (or as an alternative to) including a protective sheet, the cover layer 706 may include an encapsulation layer 800 formed from a material (e.g., TPU filaments) that is deposited along the upper surface of the conductive trace 604. The encapsulation layer 800 encapsulates the conductive trace 604 relative to the base layer 500. The encapsulation layer 800 can serve to insulate the conductive trace 604 electrically and may protect it from delamination from the base layer 500. In some configurations, the encapsulation layer 800 may extend along a majority of the conductive trace 604 (with the exception of the portions of the electrode 600 and connector 410 to make electrical connections). Alternatively, the encapsulation layer 800 may be provided only along select portions of the conductive trace 604 (e.g., along only the central portion of the conductive trace 604).

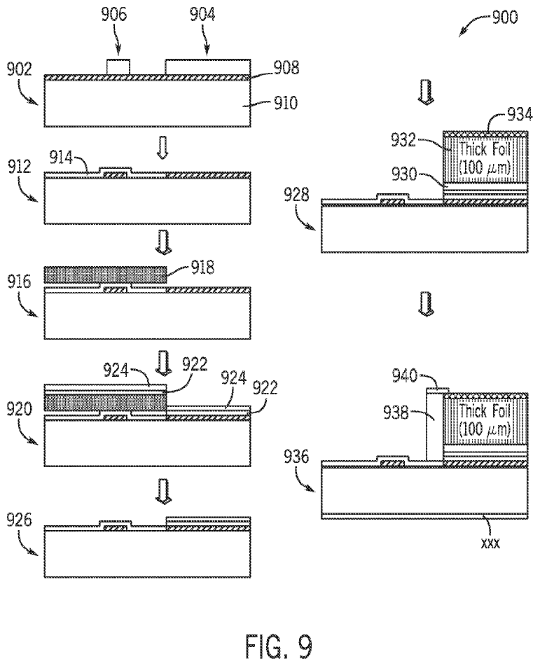

Referring to FIG. 9, a scalable approach to producing micro and nanoscale structures is provided. As will be described in this non-limiting example for producing the systems described herein and for exemplary purposes only, the structures can be formed of aluminum EEG traces with an ultra-high-aspect ratio (up to 17,000:1, with dimensions 30 nm 50.8 cm 100 μm). As will be described, long, flexible, and narrow aluminum thin film traces can be produced and incorporated into the NeoNet structure described herein.

The process 900 begins at process block 902, where photolithography of aluminum metalized polyimide can be performed. For example, an electrode 904 and trace 906 can be formed using dry photoresist arranged on aluminum 908 (e.g., 30 nm) deposited on polyimide 910 (e.g., 76.2 micrometers). At process block 912, trace insulation is performed. In particular, a layer of adhesive, such as Pyralux, (e.g., 25.4 micrometers) can be arranged to this end. At process block 916, masking can be performed by arranging Kapton tape 918 over the trace 906. At process block 920, e-beam deposition can be performed. In particular, the e-beam deposition can be performed to deposit chromium 922 (e.g., 10 nm) and gold 924 (e.g., 300 nm). At process block 926, wet etching is performed. Then, at process block 928, electrode assemble is performed where a conductive epoxy 930 can be used to apply a foil 932, which may be silver and may be thick (e.g., 100 micrometers). On the foil 932, sliver chloride 934 can be applied (e.g., 100 micrometers). Finally, at process block 936, electrode waterproofing can be performed, for example, with the application of a water-tight epoxy 938 and adhesive, such as Pyralux, 940 (e.g., 25.4).

The aluminum thin films and alloys comprise most of the interconnections used in semiconductor chips. Thin film is a technology for fabricating nanoscale electric traces and circuits. Aluminum thin films can be advantageously used in nanoscale stacked transistor fabrication at room temperature in the construction of a neonatal EEG net (NeoNet), which enables working with polymer substrates or papers, avoids the use of rare-earth elements, and produces no high-temperatures toxic by-products. Since EEG leads may act as an antenna and may capture RF waveforms induced by the MRI, may generate B1 artifacts, and may introduce safety issues, a stripline design for MRI compatibility may be created that minimizes the current flow at RF. For example, for a 3T MRI system, this equates to 128 MHz, while maintaining the SNR intact at low frequency (<1 kHz). Ideally, materials with high μᵣ would exhibit such behavior. However, metals with high μᵣ will also produce significant MRI susceptibility (i.e., B₀) artifacts. Thus, traces with low conductivity that make traces of 10 kΩ (10 kΩ resistors are inserted between leads and EEG electrodes in commercial EEG/MRI sets) of resistance or more may be preferred. Aluminum is again a good candidate since it has a high bulk resistivity (i.e., 2.65 10-8 Ωm) among all of the non-alloy, non-brittle, biocompatible, and non-magnetic metals (e.g., chromium is antiferromagnetic at room temperature). Aluminum is also the least dense (i.e., 2.65 g/cc) non-brittle elemental metal (e.g., titanium is almost twice as dense as Al at 4.5 g/cc), which confers Al with a low CT artifact material property. Finally, mechanical stress can occur if the traces are potentially pulled, especially in pediatric applications, and of course, we want the traces to remain intact both electrically and mechanically. The tensile properties of free-standing electron-beam-deposited aluminum thin films have been studied, where a high oy ductility in terms of large % elongation and ultimate tensile strength (UTS) was observed mainly due to the fine grain sizes. The Young's modulus (measuring the deformation when unloading-reloading), albeit less important, was lower than half of the reported value for pure aluminum in the bulk form.

Thus, the lead assembly 200 formed of the above-described lead systems 400 are provided. Overall, the thin-film deposition and trace-forming processes may be used for manufacturing, which advantageously minimizes the dimensions of, and amount of conductive material utilized to form each lead system 400. In particular, the high-tolerance thin-film deposition process used to deposit the trace-forming material which forms the conductive trace of the lead system 400 minimizes the thickness (i.e., height H4) of the conductive trace as compared to a height that would otherwise define the conductive trace were it formed utilizing other technologies (e.g., using thick-film technologies). Additionally, the thin-film deposition process used to form the conductive trace may be used to deposit trace-forming material on thin substrates, thereby allowing the base layer also to be defined by a thin profile (i.e., a reduced height H1), thus further decreasing the overall height of each lead system 400. This reduced thickness (i.e., overall height) of each lead system 400 may render it more flexible and thus more capable of adapting to the contours of a patient's head. This increased ability to adapt to the contours of a patient may enhance the ability of the whole lead assembly 200 to remain fixedly secured relative to the target portion of the patient during the EEG procedure and thus may minimize the risk of inadvertent movement (e.g., partial or entire detachment) of the lead assembly 200 relative to the target portion of the patient.

Similarly, the high-tolerance trace-patterning step used to define the actual width W5 of the conductive traces 604, 606 also advantageously minimizes the overall width W3 of the conductive trace as compared to an overall width W3 that would define the conductive trace where it formed utilizing other technologies (e.g., using thick-film technologies). As described above, the reduced overall width W3 of the conductive trace may advantageously allow the width W4 of the central portion and proximal end of the base layer 500 to be minimized, thereby decreasing the overall width of the lead system 400.

Additionally, in some configurations in which the conductive trace 604, 606 is defined by a trace pattern including one or more portions arranged adjacent to one another (such as, e.g., the serpentine trace pattern described above), the high-tolerance trace-patterning step advantageously also allows for adjacent portions of the conductive trace to be reliably (i.e., in a manner that avoids electrical shorting between adjacent portions) formed closer to one another (i.e., minimizes the spacing W6 between adjacent portions of the conductive trace) as compared to the spacing that would be needed were the conductive trace formed utilizing other technologies (e.g., using thick-film technologies). By allowing adjacent portions of the conductive trace to be spaced closer to one another reliably, the high-tolerance trace-patterning step thus allows for the conductive trace to be formed into trace patterns having a higher density (i.e., trace patterns that are more compact and thus defined a narrower overall width W3) than would otherwise be possible. The combined narrower actual width W5 and the increased compactness (i.e., narrower overall width W3) with which the conductive trace may be formed in such embodiments thus advantageously minimizes the footprint of the conductive trace, which may advantageously decrease the overall width of the lead assembly.

As will be appreciated, the reduced dimensions with which the conductive trace 604, 606 may be formed and/or the higher density by which the trace pattern of the conductive trace may be defined may provide a number of advantages. As an initial matter, the reduced dimensions of the conductive trace advantageously reduce the amount of conductive material contained by the lead assembly, thus minimizing the risks to patient safety and adverse impact on signal quality posed by the presence of conductive materials while obtaining EEG signals during an MRI procedure. Additionally, the reduced amount of conductive material contained by the lead assembly may also minimize the costs of manufacturing the lead assembly. The reduced dimensions of the conductive trace and/or the higher compactness with which the conductive trace may be formed may also advantageously reduce the overall height and width dimensions of the lead assembly, which in turn may also advantageously reduce the weight of each lead system 400 and, thus, the overall lead assembly 200.

One example of a benefit of the reduced amount of conductive material contained by the lead system 400, as well as reduced weight and size of the lead system 400, may facilitate the ability to acquire high-density EEG readings (e.g., 128-channel readings or even 256-channel readings) during an MRI procedure from pediatric patients, for whom such readings may not be otherwise possible due to the large size and heavy weight of conventional EEG leads. In one non-limiting example, the increased density with which the trace-forming process allows the conductive trace 604, 606 to be formed may advantageously be utilized to incorporate a plurality of discrete (i.e., independent) conductive circuits along a single base layer 500 to provide a multi-array lead assembly 200. Because of the ability of the trace-forming process to form conductive traces defined by highly compact trace patterns, the multiple conductive circuits of such a multi-array lead assembly 200 may be formed along with a base layer 500 that is nevertheless defined by a relatively narrow width. By, thus, combining multiple conductive circuits into a single lead assembly 200 having a narrow width, the trace-forming process described herein may advantageously reduce the number of lead system 400 needed to obtain high-density (e.g., 128-channel, or even 256-channel) EEG readings. The reduced number of lead systems 400 needed to be supported by the carrier assembly 208 to obtain high-density EEG readings in such embodiments may advantageously decrease the time and complexity of the necessary preparation for an EEG procedure and may reduce errors in the placement of the lead assemblies 200 during the preparation for the EEG procedure, and may reduce the risk of strangulation during the EEG procedure (e.g., during EEG procedures performed on pediatric patients).

Another non-limiting advantage of the lead assembly 200 is that the decreased size of each lead system 400 may allow for higher density acquisition of patient signals during an EEG procedure (e.g., during a combined EEG/MRI procedure). Notably, the amount of signals that may be obtained during an EEG procedure may be constrained by the physical dimensions of the head of a patient relative to which the EEG leads may be secured, the size of the EEG leads utilized to acquire signals from individual signal acquisition locations about the head of the patient, and the adverse effect that the presence of increased amounts of conductive material may have on patient safety and/or signal quality during a combined EEG/MRI procedure. Accordingly, by minimizing the size of each lead system 400, the trace-forming processes described herein advantageously allow an increased number of lead system 400 to be incorporated into lead assembly 200 for use in a constrained physical space, which in turn allows for patient signals to be obtained from an increased number of signal acquisition locations. Furthermore (as described above and as evidenced by the test results discussed below) because the trace-forming processes described herein controls, reduces, or minimizes the amount of conductive material contained by the conductive traces, increasing the amount of lead system 400 used during an EEG procedure may be accomplished in a manner that does not compromise patient safety and/or which does not adversely affect the signal quality (e.g., does not introduce noise artifacts into the obtained EEG signals). Thus, the ability to acquire a higher density of patient signals during an EEG procedure—while avoiding adverse effects on patient safety and/or signal quality—may advantageously improve the quality of the EEG results obtained using the lead assemblies 220 described herein.

Another non-limiting advantage provided by the lead assembly 200 and by the trace-forming process used to form the lead system 400 described herein is that the reduced size and minimal amounts of conductive material contained by the lead system 400 may facilitate the use of the lead assembly 200 in conjunction with one or more additional sensor assemblies. For example, as described above, movement (e.g., as a result of patient movement and/or physiological movement due to blood flow) during a combined EEG/MRI procedure may introduce movement artifacts that adversely affect the quality of obtained EEG signals. Accordingly, decreasing the dimensions of the lead system 400 makes greater space available in the carrier assembly 208 for the incorporation of one or more motion sensors into the lead system 400 without the need to sacrifice the amount of lead assemblies 200 (i.e., the reduced size of the lead system 400 may allow the incorporation of motion sensors into the carrier assembly 208 without the need to remove EEG leads to accommodate the added motion sensors).

Even more advantageously, the highly compact manner with which the conductive trace 604 of the lead system 400 may be formed may advantageously allow the lead system 400 to additionally incorporate a second conductive circuit, such as the coil/loop 602 and conductive trace 606 of FIG. 6. The coil/loop 602 may be used, for example, to acquire motion data, such as when using the lead assembly 200 within an MRI environment by acquiring MR data or by detecting the magnetic gradients. The combined acquisition of motion and physiological signals via the lead system 400—and in particular, the acquisition of motion and physiological signals using a lead assembly 200 in which a first conductive circuit (i.e., a physiological signal acquisition circuit) and a second conductive circuit (i.e., a motion signal acquisition circuit) are rigidly fixed relative to one another via their integration into a single structure—may allow for the acquisition of motion data that is highly localized and specific to the location from which a patient signal is obtained. Motion measurements highly correlated to the site from which patient signals are obtained may increase the accuracy with which motion artifacts may be filtered from the obtained patient signals and thus may improve the overall accuracy of the final EEG signals obtained by the lead assembly 200. Additional information regarding a motion sensor that may be integrated into the lead assembly 200 is provided in co-pending U.S. application 63/363,992, titled “SYSTEMS AND METHODS FOR ELECTROPHYSIOLOGICAL SIGNAL RECORDING AND POSITION OR MOTION MONITORING DURING MAGNETIC RESONANCE IMAGING,” and filed on May 2, 2022, and PCT application PCT/US2023/066485, filed May 2, 2023, the entirety of which is incorporated by reference herein.

### Studies

In one study, the NeoNet was used with CT image quality testing, MRI safety, and image quality studies, and a series of electromagnetic simulations to estimate the MRI quality of a 29-month-old child wearing the NeoNet using a finite-difference time-domain (FDTD) method. The results of the thin film-based EEG trace (NeoNet) were compared with NoNet, CuNet (without resistors), and CuNet (with ideal current limiting resistors) in terms of the B1 transmit field distortion.

A CT scan reconstructs an image of the human body by combining the photon incident counts produced by X-ray generators at different angles and by taking the negative logarithm, yields samples of the Radon transform of the linear attenuation map. However, such photons are scattered and/or attenuated by the presence of metals inside or outside the body such as EEG leads, producing hardening or streak artifacts in regions potentially of interest (e.g., brain lesions). Metal artifacts may decrease lesion identification for radiation diagnosis and reduce the accuracy of radiotherapy's target delineation and dose calculation. In order to reduce the presence of hardening artifacts, we adopted our twin approach of using a very thin metal film (i.e., 30 nm) and selected aluminum deposition, given its very low mass density:

| | Conductivity | Resistivity | Mass Density | α |
|---|---|---|---|---|
| | (S/m) | (Ω · m) | (Kg/m³) | (Ω/C) |
| Aluminum | 3.7 × 10⁷ | 2.65 × 10⁻⁸ | 2700 | 4.3 × 10⁻³ |
| Copper | 5.96 × 10⁷ | 1.68 × 10⁻⁸ | 8930 | 3.9 × 10⁻³ |
| Gold | 4.1 × 10⁷ | 2.44 × 10⁻⁸ | 19,302 | 3.7 × 10⁻³ |
| Titanium | 1.8 × 10⁶ | 5.56 × 10⁻⁷ | 4500 | 4.7 × 10⁻³ |

Aluminum was chosen as it is flexible, non-toxic, and, most of all, it is non-magnetic. Thus, aluminum will produce only minimal magnetic susceptibility artifacts. Thin aluminum film traces were selected as we will show that they are also capable of producing real and imaginary losses to reduce the antenna effect and generate the desired MRI cloaking effect of the EEG traces, thanks to the relatively low conductivity.

For simplicity, we initially studied the geometry of a birdcage coil and a thin conductive rectangular trace with length l, width w, and thickness t, oriented along the z-axis at a distance r from the center and at an angle φ from the x-axis. The MRI coil produces a rotating radiofrequency B₁ time-varying field, which induces in the trace a current along the trace that may introduce artifacts in the B₁ field according to the following one-dimensional wave equation:

$$\frac{{\partial }^{2}}{\partial {z}^{2}} {A}_{z} ( z ) = ( {ε}_{r} - \frac{j}{ω {ε}_{0} ρ} ) {k}^{2} {A}_{z} ( z ) ; \qquad Eqn . 3$$

where ρ is the resistivity [Ωm] and ε is the electrical permittivity of the bulk conductive trace, k=ω\sqrt{ε, μ₀} is the wave number of free space, and A_{z}(z) is the magnetic vector potential with the solution of Eqn. 3:

$${A}_{z} ( z ) = \frac{{μ}_{0}}{4 π} {\int }_{- 1 / 2}^{1 / 2} {J}_{z} ( z ) \frac{{e}^{- jk} \sqrt{{( z - {z}^{′} )}^{2} + tw}}{\sqrt{{( z - {z}^{′} )}^{2} + tw}} {dz}^{′} . \qquad Eqn . 4$$

The current density induced by the MRI coil is:

$$Jz ( z ) = - j ω ( \frac{1}{ρ} + j ω {ε}_{0} {ε}_{r} ) {A}_{z 1} ( z ) = - j ω σ * {A}_{z 1} ( z ) . \qquad Eqn . 5$$

For a birdcage coil with a radius R, the z-axis magnetic vector potential generated is given by the following Fourier series:

$${A}_{z 1} ( z ) = {\sum }_{m} \frac{{μ}_{0} {S}_{m ( z )}}{2 \vert m \vert} {( \frac{r}{R} )}^{\vert m \vert} {e}^{{jm}_{φ}} ; \qquad Eqn . 6$$

where Sₘ are the surface currents along the birdcage coil. Therefore, the magnetic vector potential is:

$${A}_{z} ( z ) = \frac{- j ω σ * {µ}_{0}^{2}}{8 π} {\sum }_{m} \frac{{e}^{jm}}{\vert m \vert} {( \frac{z}{h} )}^{\vert m \vert} {\int }_{- 1 / 2}^{1 / 2} {S}_{m} ( z ) \frac{{e}^{- jk \sqrt{{( z - {z}^{′} )}^{2} + tw}}}{{( z - {z}^{′} )}^{2} + tw} {dz}^{′} = - \frac{j ω σ * {µ}_{0}^{2}}{8 π} {Γ}_{z} . \qquad Eqn . 7$$

Thus, the B₁ₜ artifact induced by the trace is:

$${B}_{1 t} = - \frac{j ω σ * {µ}_{0}^{2}}{8 π} ∇ \times {Γ}_{z} . \qquad Eqn . 8$$

This is reduced by reducing σ*, or equivalently by reducing the relative permittivity (however εᵣ=1 in metals) and increasing the trace resistivity, which can be achieved by selecting metals with higher resistivity. The total trace resistance increases linearly with the metal resistivity:

$${R}_{t} = ρ \frac{1}{wt} . \qquad Eqn . 9$$

However, in order for Rₜ≥10 kΩ, resistors were added at the end of the trace that are commonly used by the industry for safety, whereas the aluminum traces required a nanoscale thickness to avoid the need for additional resistors. Several studies have shown that the resistivity of aluminum conductors may increase just by reducing one dimension to the nanoscale, which is a desirable property since too thin a width in the traces may have manufacturing and reliability issues. The hypothesis behind this physical property was first proposed in 1938 by Fuchs, and this effect occurs when the nanoscale thickness of the metal is shorter than the mean free path of the conduction electrons, resulting in collisions with the boundaries of the film. The numerical model that predicted the thin film resistivity was subsequently improved to correctly describe the resistivity changes with thickness in the case of different film morphologies (e.g., mono, polycrystalline films, etc.), and thus various types of electron scattering (e.g., background, grain boundaries, and surface scattering).

According to the combined Fuchs-Sondheimer and Mayadas-Shatzkes conduction model (MS-FS), the ratio between the bulk and thin film resistivities is:

$$\frac{{ρ}_{0}}{ρ} = 1 - \frac{3}{2} α + 3 {α}^{2} - 3 {α}^{3} Log ( 1 + \frac{1}{α} ) - f ( k ) . \qquad Eqn . 10$$

where ρ₀=2.65·10⁻⁸ [(Ω·m] (i.e., bulk aluminum resistivity) and:

$$f ( k ) = \frac{6 ( 1 - p )}{π k} {\int }_{0}^{π / 2} {\int }_{1}^{∞} ( \frac{1}{{y}^{3}} - \frac{1}{{y}^{5}} ) \frac{{cos}^{2} ϑ ( 1 - {e}^{- ktG ( y , ϑ )} )}{{G ( y , ϑ )}^{2} ( 1 - {pe}^{- ktG ( γ , ϑ )} )} d ϑ dy ; \qquad Eqn . 11$$

where p=0.510 is the fraction of electrons specularly scattered at the external surfaces, k is the film thickness t divided by the bulk electron mean free path l₀, and

$$( y , ϑ ) = 1 + \frac{α}{{( 1 - 1 / {y}^{2} )}^{2} cos ( ϑ )} ; \qquad Eqn . 12$$

where

$$α = \frac{{l}_{0} r}{{a}_{g} ( 1 - r )} ,$$

and a_{g} is the crystalline size and r is the grain boundary reflection coefficient. The MS-FS model can be simplified as:

$$\frac{{ρ}_{0}}{ρ} ≡ 1 - \frac{3}{2} α + 3 {α}^{2} - 3 {α}^{3} Log ( 1 + \frac{1}{α} ) - \frac{3 ( 1 - p )}{8 k} . \qquad Eqn . 13$$

According to our measurements R≅2.5 kΩ when I=0.463 [m] and w=10⁻⁴ [Ω]. Thus, the minimum thickness based on the bulk resistivity of Al and Eqn. 9 was t≅50 nm (i.e., almost twice as thick compared to the value t=30 nm reported in the datasheet of the aluminum metalized polymer film utilized), while Eqn. 12 predicted a t≅1 μm based on k≅1, and α=2.5. In order to reach the desired Rr we designed traces with 1≅2.5 [m], but given our photolithography panel size limit, we deigned traces with N=5 loops. The temperature-dependent overall resistance is:

$${R}_{t} = ρ \frac{( N + 1 ) I}{wt} [ 1 + α \cdot ( T - 20 ° ) ] ; \qquad Eqn . 14$$

where T is the temperature [° C.] and α is the temperature coefficient [Ω/° C.].

### NeoNet Construction

Sheets of 540 mm×540 mm, 50 μm thick polyethylene terephthalate polyester (PETP) film already vacuum coated on one side with 30 nm aluminum were used (ES301955, GoodFellow, Coraopolis, PA, USA). The traces were composed of seven process fabrication steps described with respect to FIG. 9. The traces were manufactured (TechEtch Inc., Plymouth, MA, USA) using photolithography, etching, pressure-mounting polyimide dielectric films, and laser cutting (e.g., process block 902 and 912). Since the electrodes were supposed to be in contact with the KCl solution and disinfectant solution that abraded the 30 nm thin aluminum, the electrodes were coated with a thin 300 nm layer of gold, with a 10 nm layer of chromium to improve adhesion (e.g., process blocks 916, 920, and 926). The NeoNet electrodes were built based on a silver/silver chloride coating of thin (25 μm) pure silver sheets by a leading manufacturer of disposable silver/silver chloride coated electrode sensors. The process consisted of coating a silver foil, 0.025 mm (0.001) thick, annealed, 99.95% (12190, Alfa Aesar, Tewksbury, MA, USA) with silver chloride (Ag/AgCl) with a chemical process. Ag/AgCl foils were cut into a disk of ¼ diameter and glued the disks to the aluminum traces using a silver conductive epoxy adhesive (MG Chemicals, ON, Canada) and embedded in a custom-made holder (Brain Products, Germany) in contact with a sponge.

### MRI Safety Recordings

The RF safety of the NeoNet was tested in a 3 T MRI (Prisma, Siemens Healthineers) using a 29-month-old head-sized agar phantom. The target dielectric properties were chosen to match the averaged pediatric brain and skin properties at 128 MHz (εᵣ=74.95, σ=0.64 (S/m). The MRI dielectric phantom recipe generator was used to estimate the recipe for the target dielectric properties. The two test samples were obtained by mixing 1 L of distilled water (DI) with NaCl (purity 98%, Sigma-Aldrich Cop, MO), Sugar, and edible agar-agar powder (Golden Coin Agar Powder, Capital Food International, Inc., La Mirada, CA, USA). The dielectric properties were measured using a network analyzer (ENA series, Keysight, Santa Rosa, CA, USA) with a high-temperature dielectric probe (85070E Kit, Agilent Technologies, Santa Clara, CA, USA). A sample of 51.3 mM saline water was used to check the measurement accuracy. The phantom's mold was 3D printed (Figure S7) in MARTIN's head's shape. Finally, the mold was filled with agarose solution, cured for at least 24 h, and stored in an MRI scanning room for 48 h for thermal stabilization. The 8-channel fiber optic probes (OSENSA Innovations Corp., Coquitlam, BC, Canada) were positioned at distributed locations across NeoNet including three hot spots estimated from the thermal simulation [11]. A thermal paste was used to allow the fiber optic probes to be in contact with the surface of the agar phantom and the EEG electrode to assess the RF-induced heating by the EEG net. A high-power turbo spin-echo sequence (21 slices, 0.9 0.9 5.0 mm voxels, TR/TE=7600/86 ms, FA=120°, Average: 20) that delivered 100% SAR for 30 min (SARhead: 2.85 W/kg, 10 gSARtorso local: 9.99 W/kg) was set to produce the maximum allowed RF safety limit in the clinical scan (IEC 60601-2-33:2010).

### CT Quality Recordings

The CT compatibility test was conducted on a head phantom acquired in the Department of Radiology at MGH using a Siemens SOMATOM Force (Siemens Healthineers, Forchheim, Germany). The images were acquired using the Pediatric Routine Head protocol (100 kVp, eff. 66 mA, 1 s rotation time, 0.8 pitch, 35 cm DFOV, 50 cm SFOV, Hr59 h kernel) performed on NeoNet while the net was positioned on a child head-sized agar phantom described above.

### MRI Quality Recordings

We imaged the three subjects in a 3 Tesla MRI (Siemens Healthineers, Germany). Since there are no commercially available pediatric nets with a label that permits scanning using the proposed clinical sequences, we used only two conditions in counterbalanced order: (1) No-Net and (2) NeoNet. Two board-certified neuroradiologists (ML, RG, each with greater than 20 years of experience reading clinical CT and MRI scans) evaluated the overall image quality of the paired NeoNet and NoNet images (three T1-weighted images, one T2-weighted image, and one diffusion tensor image), blinded to the presence or absence of the NeoNet device. A five-point Likert scale was used to score the image artifact, overall image quality, and clinical usability as follows: 1, extremely poor image quality (major artifacts exist and the images are not clinically useful); 2, poor image quality (major artifacts exist and clinical use is not advised); 3, average image quality (average moderate artifact detected, but minor consequences to clinical use); 4, good image quality (containing minor artifacts which do not adversely affect the clinical use); 5, excellent image quality (no artifacts). Images were pre-processed to remove the EEG electrodes visible outside the skull for blinding purposes. We used Infant Freesurfer to perform skull stripping and create a brain mask on the T1- and T2-weighted MR imaging and diffusion tensor imaging (DTI) scans for both the NoNet and NeoNet recordings of all subjects. Subsequently, we removed all the structures that were located outside this brain mask including the skull, skin, and electrodes. The new images were randomly shown to the neuroradiologists (ML,RG) who were unable to see the presence/absence of electrodes on the skull by the masking process and were asked to score the quality of each scan based on the Likert scale, as described above. Finally, the scan ratings were compared by using a two-tailed paired t-test using SPSS software (IBM, Armonk, NY, USA). Results are reported as (Median, IQR), and statistical significance was defined as p-value<0.05.

### Numerical Simulations

Sim4Life (ZMT MedTech, Zurich, Switzerland) was used to solve Maxwell's equation at 128 MHz using the FDTD method. The shielded birdcage body transmit coil was designed following the realistic dimensions of a 3T MRI with a diameter of 610 mm, a spoke length of 670 mm, a shield diameter of 660 mm, and a length of 1220 mm. The coil was driven in the circularly polarized (CP) mode. The head of the 29-month-old male whole-body voxel model was positioned at the center of the body coil to assess the complex B1 field distribution. Masks were applied on the surrounding air to display the B1 transmit field difference in the child model, which would otherwise not be visible because of scaling issues.

The 128-channel EEG traces were modeled following the scalp from the temporal lobe toward the parietal lobe, exiting through the top of the head. The 1 mm×1 mm trace width/thickness and dielectric properties (εᵣ=4.2, σ=41.7 S/m) were chosen to represent 12.23 kΩ as the nominal trace thickness was too thin to be represented in the model (i.e., 30 nm). Each trace was designed to have a minimum distance of 1 mm from each other as well as from the skin to avoid contact between the traces and the skin. A locally dense grid was applied along the traces with a resolution of 0.7 mm×0.7 mm×1.0 mm. A high-performance graphics processing unit (GPU) (NVIDIA V10, Nvidia, Santa Clara, CA) was used to reduce the computation time. The B1 transmit fields were computed in four different scenarios: (i) a pediatric model without an EEG net (NoNet); (ii) with a thin film-based EEG net (NeoNet); (iii) with copper traces with an ideal current limiting resistor (R=10 kΩ) between sponges and copper traces (CuNet); (iv) with copper traces without current limiting resistors. The B₁ transmit field and current density was normalized to a field that produced 2 μT at the center of the coil in the case of NoNet. Then, the same input currents were applied to all other cases. The B₁ transmit field and current density map were compared to estimate the effect of the different types of EEG traces. The difference of |B₁⁺| between NoNet and (i) NeoNet, (ii) CuNet (copper traces with an ideal current limiting resistor), and (iii) CuNet (copper traces without current limiting resistors) were calculated as:

$$Δ \vert {B}_{1}^{+} \vert = mean ( \frac{{\vert {B}_{1}^{+} \vert}_{NeoNet or CuNet} - {\vert {B}_{1}^{+} \vert}_{NoNet}}{mean ( {\vert {B}_{1}^{-} \vert}_{NoNet} )} \times 100 ) head ; \qquad Eqn . 15$$

where mean (|B₁⁺|_{NoNet}) is the mean value of |B₁⁺| in the case of NoNet over the volume of the head. The sensitivity factor of each parameter was calculated by running two simulations that differed only by a single parameter value of the dielectric properties of the muscle, skin, and subcutaneous adipose tissue (SAT) or fat compartments close to the EEG electrode. The second value (Value2) was set with a 10% change in the dielectric.

### EEG Recording

The EEG recording was performed using a commercial High-Density (128 channel) EEG system (The Magstim Company Limited, Whitland, United Kingdom) and the NeoNet EEG systems on a pediatric patient with epilepsy. As per standard clinical practice, 20-40 min of EEG recordings with the commercial net were completed and followed by 10 min of EEG recording with the NeoNet EEG. One ECG electrode incorporated into the EEG cap performed the ECG recordings to prevent the ballistocardiac artifact of the EEG. A pediatric clinical neurophysiologist (J.P.) performed a qualitative comparative review of both EEG recordings to assess the quality of the background pattern and identify physiological and pathological (epileptiform) graphoelements.

### Results

### NeoNet Fabrication

The thin film traces were fabricated, and the resistance resulted in a high resistance of approximately 12.23 kΩ at room temperature (i.e., 20° C.), with a minimum standard deviation (SD) of 0.49 kΩ, with a target resistance between 10 kΩ and 15 kΩ. The target size of the trace width, w=100 μm, was based on the coupon testing and the failure rates, which increased approximately inversely proportional to w.

### MRI Safety

MRI safety testing confirmed these findings with a 30-min scan, and the maximum temperature rise was found to be 0.84° C., which is the condition that allows for 1 h of scanning without cooling time in normal operating mode as per the FDA guidelines.

### Image Quality

A qualitative comparison of the MR images showed a similar image quality with NoNet and NeoNet, as predicted by our previous numerical simulations. Images of NoNet and NeoNet did not show any metal artifacts. No artifact from the aluminum thin film-based leads was visible.

### Numerical Simulations

The axial, coronal, and sagittal view of the absolute B1 transmit field was compared. The difference was considered in the absolute B1 transmit fields between (i) NoNet vs. NeoNet, (ii) NoNet vs. CuNet with ideal current limiting resistances, and (iii) NoNet vs. CuNet without resistors compared in the axial, coronal, and sagittal views. The phase of the B₁ transmit field in the axial, coronal, and sagittal views in the case of NoNet, NeoNet, and CuNet with ideal current limiting resistors and CuNet without resistors was considered. The difference in phase of the B₁ transmit field between (i) NoNet vs. NeoNet, (ii) NoNet vs. CuNet with ideal current limiting resistors, and (iii) NoNet vs. CuNet without resistors was also compared. The studies also considered the different magnitude and phase B₁ transmit field plots, especially the difference plots, show that NeoNet had the smallest changes compared to the NoNet case, followed by the case of CuNet with a current limiting resistor, and finally without current limiting resistors. The FDTD simulations estimated a peak root mean squared (RMS) current density of 260 kA/m² in the case of CuNet without resistors, 218 kA/m² in the case of CuNet with resistors, and only 9 kA/m² in the NeoNet case. The NeoNet had the smallest Δ|B₁⁺| compared to the NoNet case, followed by the case of CuNet with a current limiting resistor. In contrast, the case of CuNet without current limiting resistors showed the largest Δ|B₁⁺| compared to the case of NoNet, as expected.

### EEG Recording

We recorded the EEG data on a 5-year-old male pediatric patient (Subject 4) with refractory focal epilepsy, developmental delay, and behavioral concerns. Complex partial and tonic-clonic seizures characterized his epilepsy. We recorded 10 min of resting-state brain activity with a commercial HD-EEG and the NeoNet and compared the two recordings qualitatively. Critical clinical information was detected with the NeoNet and commercial EGI HD-EEG net. The same subject of the left frontotemporal interictal epileptiform discharges was identified in both the commercial HD-EEG and NeoNet recordings. Furthermore, physiological features such as muscle artifacts and sleep spindles were present, while eye movement artifacts were identified in the frontal leads. Brief, morphologically defined events characteristic of patients with epilepsy such as interictal epileptiform discharges were also identified.

### Discussion

### NeoNet Fabrication

We found that the proposed thin film fabrication was superior to a previous prototype, which was manufactured using polymer thick film (PTF) with a similar technology. The trace resistance was 12.23 kΩ for the thin film fabrication of 128 traces with a SD of 0.49 kΩ whereas the resistance was found to be, on average 20.49 kΩ, with a larger SD of 0.85 kΩ for 256 traces made with PTF technology. This disparity between the target and measured resistance was because the trace resistance was achieved by mixing pure silver and carbon inks. The final resistance variability depends on many factors including the mixing ratio and variables hard to control such as environmental temperature and humidity, oxidation of the mixture, and wettability of the materials, and it can only be found on the manufacturing day by test and retest. Finally, the SD for a larger number of 256 vs. 128 traces should be lower. Instead, it resulted in a larger SD for the thick film technology.

### MRI Safety

The use of conductive EEG leads in the presence of a radio-frequency field generates induced currents on the leads and potential increases in RF power absorbed in the human head, specified regarding the specific absorption rate (SAR). For relatively high-power RF sources such as MRI RF coils, such interactions pose serious thermal-related safety risks of tissue heating and burns. Finite difference time domain numerical estimations using a high-resolution model of head tissues with EEG leads during MRI scanning simulation showed the presence of local SAR peaks near the EEG electrodes, with values depending on a wide range of variables, namely, the lead orientation shielding effect of EEG leads, RF frequency, number of EEG electrodes/leads, and resistivity of the EEG leads. These simulations, followed by temperature measurements during MR scanning with an anthropomorphic phantom, are essential for establishing system safety. Numerical simulations and temperature measurements conducted in such studies indicated that the use of metallic leads during MRI should be avoided and that resistors (currently, 5-15 kΩ is the industry standard) placed on the leads do not offer reliable protection against the antenna effect of the leads exposed to the MRI RF-field. In contrast, the increased lead resistivity afforded by PTF/thin film technology allows for safe and high-quality recordings up to 7 T. With regard to EEG-fMRI safety in infants/toddlers, we could only find a single study that found that on a phantom, an MRI sequence (T2 with Max Turbo factor 25; SAR 89%) caused a large temperature increase at one electrode (Fpz; +4.1 C). Additionally, this study concluded “Based on our findings, we strongly recommend against using the structural images obtained during simultaneous EEG-MRI recordings for further anatomical data analysis”. Our previous studies showed the MRI RF safety of the NeoNet using numerical simulation. This study showed that the high-power MPRAGE heating was well below 2° C. Our recent simulation study on EEG safety with an infant/toddler also showed a similar finding based on the dielectric properties of the resistive traces in infants/toddlers for safe EEG-MRI, where thin film resistive traces reduced the RF heating at 3 T MRI within the safety guidelines by the FDA. Thus, resistive traces may improve the safety of infant/toddler EEG-MRI, as shown by our SAR, and thermal safety, regardless of the sequence used. In this study, our target trace resistance of 10-15 kΩ was based on our recent study in which we studied the safety using resistors of 10 kΩ, and the 5-15 kΩ industry standard. A lower resistance below 10 kΩ may affect the amount of RF-induced currents in the NeoNet traces inside a 3 T MRI, which could lead to both heating and greater B₁ artifacts. For very total high resistances of the trace resistance and the contact EEG resistance on the subject, the EEG quality may be compromised and may damage the amplifier (the maximum total resistance allowed may vary depending on the manufacturer).

### Numerical Simulations

Since all of the materials used in the fabrication of the NeoNet did not interfere with the B₀ field (i.e., no susceptibility) nor produced a chemical shift artifact, we studied the B₁ transmit field distortion on a 29-month-old male model wearing 128-channel HD-EEG nets in a 3 Tesla MRI. The numerical simulations predicted the observed artifact-less MR images with the NeoNet, namely, the FDTD algorithm predicted a significant differential increase (with the NoNet) in the magnitude of the B₁ transmit field of 65.2% for the CuNet with resistors and 137.5% for the CuNet without resistors compared to the NeoNet. The electromagnetic (EM) simulations were conducted on a child model to estimate the B₁ transmit field distortion in the case of resistive EEG traces (NeoNet), CuNet with ideal current limiting resistors, and CuNet without current limiting resistors. Additionally, current density maps on three different EEG trace conductivities were compared to estimate the amount of current induced on each EEG trace due to the antenna effects. The CuNet without a resistor induced a 29-fold peak RMS current density increase over the NeoNet, whereas the CuNet with ideal current limiting resistors induced a 24-fold peak RMS current density increase over the NeoNet. Therefore, the current-limiting resistor does not effectively reduce the induced current in the lead. The resulting effect is that B₁ field distortions that these currents generate (Ampere's law) are not affected in practice by “current limiting” resistors. In the case of CuNet without current limiting resistors, the overall B₁ transmit field on the pediatric head was reduced due to the shielding effect. Nevertheless, the extremely low B₁ transmit field interaction of the NeoNet indicated an MRI signal almost identical to the NoNet case, which stemmed from the reduced current density induced in the resistive traces compared to the copper traces.

### MRI, CT, and EEG Recordings

Two board-certified neuroradiologists (R.G. and M.H.L., with more than 20 years of clinical and neuroimaging research experience, respectively) independently scored the quality of MR images. Comparing the images of different sequences (T1, T2, and DTI) with NoNet and with NeoNet showed no difference in the Likert score for Subjects 1-3. Subject 1 was selected as a healthy adult with a small head circumference who reported that the NeoNet was comfortable enough for studies in children.

Metallic electrodes and leads may partially or entirely block X-rays. The nonlinearity caused by this phenomenon leads to a streak of low and high attenuation in the image, obscuring the underlying anatomy. Such image artifacts may obscure lesions such as small metastases. Aside from lesion identification, metallic implants may interfere with radiation delivery by blocking it from reaching a deeper target. No quantitative Likert score was performed for CT scans since neuroradiologists are not used to evaluate phantom images. However, the absence of CT artifacts with the NeoNet was evident in all slices due to the low-density materials (e.g., aluminum) used in the NeoNet that avoided streak artifacts.

Regarding EEG metal artifacts, an increase in signal intensity as a B₁ artifact predicted by the simulations was shown. In contrast, a decrease in signal intensity as a B₁ artifact predicted by the simulations was not. In part, these artifacts could also be susceptibility artifacts due to the different magnetic properties of the metals with respect to the human tissue.

Comparative EEG data recorded with a commercial HD-EEG net and the NeoNet showed a similar quality in both recordings. Physiological EEG features were clinically identified in both recordings by a neurologist (J.M.P.), and the same population of interictal epileptiform discharges was identified in the recording of the same subject, in support of the clinical utility of the NeoNet EEG signal compared to commercially available net(s).

### CONCLUSIONS

Metallic artifacts and distortions can substantially degrade the image quality of both MRI and CT when EEG electrodes are present. Thus, we showed that the novel thin film trace EEG net (“NeoNet”) resulted in improved MRI and CT image quality without compromising the EEG signal quality. The 50 cm long thin film trace was constructed from a 30 nm aluminum film, resulting in low density for reducing CT artifacts and a 12 kΩ high-resistive low for reducing MRI artifacts. The NeoNet demonstrated safety in 3 Tesla (T) MRI for children with an increase of just 0.84° C. after 30 min of high-power scanning, which is the acceptance criteria for the temperature for 1 h of normal operating mode scanning as per the FDA. We investigated the effects of EEG nets on B₁ transmit field distortion in 3 T MRI with electromagnetic simulations, which predicted a 65% B₁ transmit field distortion higher for commercially available copper-based EEG nets over the NeoNet. No significant difference in the degree of MRI artifact or image distortion was found while analyzing the Likert scale responses from two board-certified neuroradiologists blinded to the presence or absence of NeoNet. Finally, NeoNet did not impact the quality of the EEG recording and allowed 128 dense EEG spatial sampling channels for clinical epilepsy diagnostics.

The proposed NeoNet device, therefore, has the potential to allow concurrent EEG acquisition and MRI or CT scanning without significant image artifacts, facilitating clinical care and EEG/fMRI pediatric research.

Another study was conducted that included electromagnetic and thermal simulations of three different conditions for EEG electrodes, for three different electrodes, including (i) a resistive, high-definition EEG electrode (HD-EEG) assembly as described above (referred to in this study as NeoNet), (ii) a copper-based EEG assembly using a carrier instead of adhesively-applied electrodes (CUNet), and (ii) a traditional EEG electrode system without a net, such that electrodes were applied via an adhesive (NoNet). The CUNet is a commonly studied case because commercial EEG traces/wires an: composed of copper. A whole-body child model was used to assess the RF-induced heating at 3 Tesla MRI (i.e., 128 MHz Larmor frequency).

For the study, a 29-month-old whole-body voxel model, MARTIN, was used in numerical simulations to compare The EM field interaction in the three cases (NeoNet, CuNet, and NoNet) in a 3 Tesla MRI environment. The numerical model of the HD-EEG net was composed of electrodes (i.e., sponges), pedestals (not included in the model), and traces. The EEG trace in the NeoNet was resistive to minimize the RF heating during MRI. To build the HD-EEG traces, the following steps were followed:
(1) The EEG electrodes' position was determined from the 3D scanned HD-EEG net on the MARTIN's 3D printed head. Each electrode (diameter. 5 mm, length: 8 mm) was positioned perpendicularly to the mood's skin, and to avoid gaps between the model and the electrodes, these were inserted slightly into the skin. Afterward, the head's partial electrode volume was removed during the voxelization process by assigning a higher priority for the grid on the MARTIN model than the electrodes.
(2) Fifteen reference trajectories were routed through adjacent rows of the electrodes lined up from temporal, frontal, or occipital to the parietal at the distance of 10 mm on top of the skin. The initial starting point for each reference trajectory was located at the first electrode of the respective row.
(3) Each reference trajectory had a length of 510 mm and ran perpendicular to the head on top of the reference electrode, where it was spaced at least 1 5 mm apart from every other reference trajectory. This routing trajectory is typical of a type of commercial (Brain Products GmbH, Gilching, Germany) EEG nets designed to escape the wires through the head coil.
(4) Up to nine traces were grouped in 3×3 arrays routed 1 mm apart, running parallel to the reference trajectories.
(5) The 128 traces were following the reference trajectory with a width of 1 mm in diameter.

The child's head with the HD-EEG net was positioned at the center of a body transmit coil (i.e., internal diameter: 610 mm, leg length: 570 mm, endring width: 25 mm).

The dielectric properties of the HD-EEG traces for the NeoNet were chosen as σ=46.30 S/m, and relative permittivity of 4.2, while σ=5.7·10⁷ S/m, and relative permittivity of εᵣ=4.2 for the CuNet. The properties of electrodes (i.e., sponges) soaked in saline solution were chosen for σ=2.14 S/m, εᵣ=84.7, respectively. The dielectric properties of tissues were selected from the MARTIN model.

The complete set of harmonic Maxwell's equations was solved at 128 MHz using a finite difference time domain (FDTD) solver using a non-uniform Yee cell grid in Sim4Life (Zurich, Switzerland). A high-performance GPU card (Tesla V100 32 GB, NVIDIA, Medford, MA) was used to sample the traces with a grid fine enough to accurately model the geometry of the traces with a resolution of 0.7 mm×0.7 mm×1.0 mm. The total grid size, including the RF coils, EEG traces, and anatomical model simulated in Sim4Llfe, included 255.8·10⁶ Yee cells. A 16-channel high-pass birdcage coil was used to generate a B1 transmit field with circularly polarized (CP) mode with an RF shield timed to 3 Tesla Larmor's frequency of 128 MHz. The electric field strength was normalized to produce a specific absorption rate (SAR) of 3.2 W/kg averaged over the child's head in the NoNet case, which is the maximum allowed RF exposure level in a normal operation mode in the IEC guidelines. In each of the three cases, the simulations' magic time step was 0.65 ps, and the total number of steps was 596,645.

The temperature of each tissue (T) over time was estimated using Pennes' bio-heat partial differential equation:

$$ρ \cdot c \frac{\partial T}{\partial t} = ∇ \cdot ( k \cdot ∇ T ) + ρ \cdot Q - ρ \cdot W ( T ) {ρ}_{b} {c}_{b} ( T - {T}_{b} ) + ρ \cdot SAR ; \qquad Eqn . 16$$

where ρ is the tissue mass density matrix (kg/m³), ρ_{b} is the blood mass density (k/m³), c is the heat capacity matrix (J/kg/° C.), c_{b} is the blood heat capacity (J/kg/° C.), T is the temperature matrix (C), T_{b} is the basal blood temperature (C), k is the thermal conductivity matrix (W/m/° C.), Q is the metabolic heat generation rate matrix (W/kg), W(T) is the thermoregulated blood perfusion rate matrix (ml/min/kg), and SAR is the specific absorption rate matrix (W/kg). The SAR is the peak spatial SAR. The thermoregulated perfusion rate (W(T)) is patient-dependent. The thermoregulated perfusion rate was given as:

$$W ( T ) = {W}_{0} \cdot {L}_{b} ( T ) ; \qquad Eqn . 17$$

$$where : {L}_{b} ( x , y , z , t ) = \{ \begin{matrix}1 & T < {T}_{0} \\ 2 \frac{T ( x , y , z , t ) - {T}_{0}}{Δ B} & {T}_{M} > T \geq {T}_{0} , T \\ 2 \frac{{T}_{M} ( x , y , z , t ) - T}{Δ B} & T \geq {T}_{M}\end{matrix}$$

$$M = \{ \begin{matrix}45 ° C . & if ( x , y , z ) ∈ skin \\ 43.4 ° C . & otherwise\end{matrix}$$

where T₀ is the initial temperature of the basal perfusion rate (T₀=37° C.), and T_{M} is the maximum thermoregulated perfusion temperature matrix (° C.). W₀ (ml/min/kg) is the basal perfusion rate matrix below T₀, L_{b}(T) is the local temperature-dependent multiplier, and is the local vasodilation parameter to match the desired perfusion increase over temperature change above T₀ (ΔB=1.6° C.).

There is no database for children's thermal properties that were known. The perfusion rates are both age-dependent and tissue-dependent, whereas specific heat capacity and thermal conductivity are assumed in this work to be only tissue-dependent. Thus, the child tissue perfusion properties were estimated from adult data by considering cerebral and non-cerebral tissues.

The thermal simulation was conducted in a two-step process to estimate the child model's relative temperature rise with and without the two HD-EEG nets during an MRI scan with the clinically maximum allowed RF power.

### Estimating the Thermal Equilibrium in Biological Tissues

The steady-state temperature of each tissue T₀=T(x,y,z,0) depends on the boundary conditions, the different metabolic heat generation rates, and the thermal properties of each tissue. In order to find the equilibrium temperature T₀, a thermal solution was computed with a null external EM source. The equilibrium temperature T₀ was only computed once per condition (NoNet, NeoNet, and CuNet). The Dirichlet thermal boundary condition was set on the blood with a temperature of 37° C. A convection-dependent (mixed) boundary conditions were set as follows:

$$k \frac{dT}{dt} + h ( T - {T}_{AIR} ) = {F}_{0} ; \qquad Eqn . 18$$

where h=6 W/m²/° C. is the heat transfer rate for external air, whereas h=10 W/m²/° C. is the heat transfer rate for air inside the child model, T_{AIR}=23° C. is the environment temperature (i.e., MRI room), and F₀ is the null heat flux matrix.

Thermal Simulation with EM Source Using Pre-Calculated Equilibrium Temperature.

The maximum temperature (worst-case scenario) was estimated by solving Pennes' bio-heat equation 16 with perfusion rates in equation 17 using a structured time-domain solver with a stable time-step. The solution T was computed in the entire geometry and with a stop time of 15 minutes. The isotropic grid resolution was set to 2 mm×2 mm×2 mm in child and surrounding air, and a conformal thermal solver was used to minimize the stair-case artifact. The total number of cells was 522.4·10⁶.

### Uncertainty Analysis

The uncertainty analysis of the numerical simulation was performed. Each parameter's sensitivity factor was calculated by running two simulations, which differed only by the single parameter value. In the case of trace and electrodes, the electrical properties (the conductivity and permittivity) were increased by 20%, whereas in the case of tissues, the electrical properties were decreased by 10%.

### Numerical Simulation of the EM Field

The results of peak SAR (pSAR) were compared with and without the HD-EEG nets. The maximum pSAR of the NoNet was 81.39 W/kg, 806.23 W/kg for the NeoNet, and 2155.64, W/kg for the CuNet. These maximum values were observed in the proximity of the EEG electrodes for the HD-EEG nets or the neck for the NoNet. The lg-mass averaged SAR (lgSAR) was 19.04 W/kg in the case of the NeoNet, 18.51 W/kg in the case of the NoNet, whereas the CuNet case generated 65.90 W/kg.

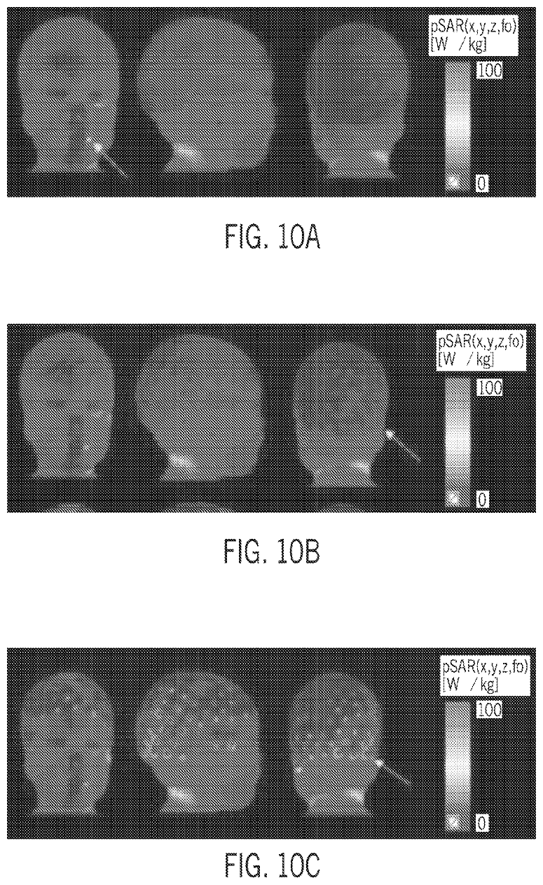

As evidenced by the test results discussed with reference to FIGS. 10A-10C. In particular, the pSAR results in the child's surface of the head, is shown in FIG. 10A with NoNet, In FIG. 10B with the NeoNet, and in FIG. 10C with the CuNet. For all simulations, the input powers were normalized to produce 3.2 W/kg in the head of the child voxel model without the HD-EEG nets. The arrows indicate the position of the maximum pSAR. As can be seen, the NeoNet delivers far superior SAR performance over CuNet.

Thermal simulations yielded similar results. The thermal simulations estimated that the maximum temperature changes in the head after 15 minutes were 36.42° C. for NoNet, 36.66° C. for NeoNet, and 42.25° C. for CuNet. The time course of maximum temperature changes yielded an exponential, as expected by the bio-heat equation, and meant that the NoNet and NeoNet performed similarly, whereas the CuNet was inferior.

This analysis shows that the numerical calculation of the peak SAR, lgSAR, and thermal elevation of 128-channel HD-EEG nets created using the systems and methods of the present disclosure (NeoNet) on an anatomically accurate 29-month-old whole-body model in 3T MRI compared to the NoNet case. This indicates that the NeoNet does not produce more than 0.2° C. heating in a 3 Tesla MRI for a 15 minutes continuous scan, whereas the CuNet show increases in the skin temperature up to 42.25° C. with a maximum increase of 6.79° C. This data with respect to the smaller and, thereby, more challenging environment of a child readily translates to adults and shows that the systems and methods of the present disclosure far outperform traditional EEG systems using the copper-based net.

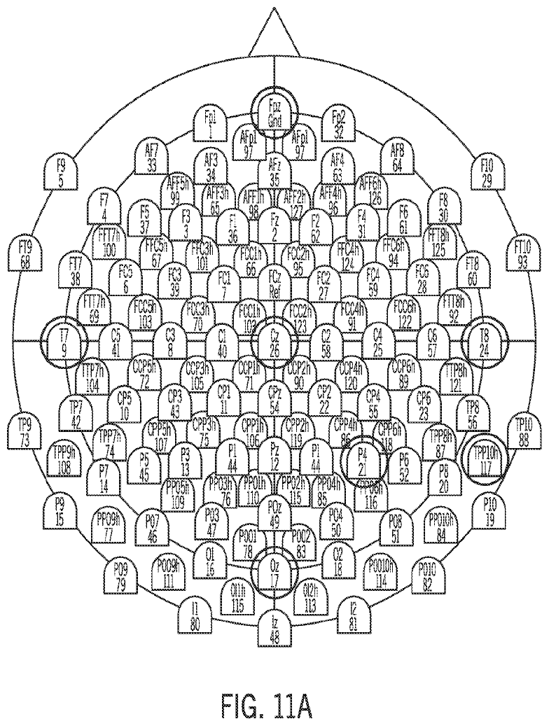

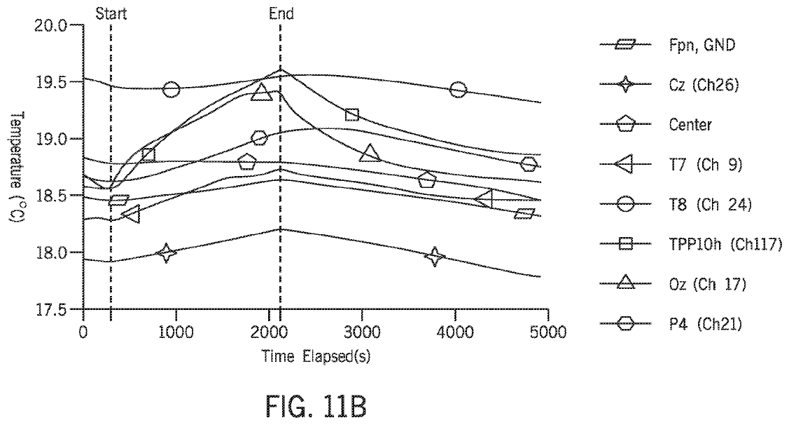

Referring to FIGS. 10A-10B, the RF safety of a carrier assembly 208 incorporating 128 lead system 400 was tested in a 3T MRI (Prisma Connectome, Siemens Healthineer) using a child head-sized agar phantom. The dielectric properties of the phantom were selected to be similar to the pediatric brain properties (i.e., σ=0.64 S/m, εᵣ=74.95). As shown by the reference map of FIG. 11A, the 8-channel fiber optic probes were positioned at the eight reference locations of the 128-channel distribution of the lead assembly 200, including three hot-spots estimated from the thermal simulation (described above with reference to FIGS. 9A-9C), and thermal paste was used to allow the fiber optic probes to be in contact with the surface of the agar phantom to assess the RF-induced heating by the lead assembly 200. A high-power turbo spin-echo sequence was set to produce 3.2 W/kg in the head, which is the maximum allowed RF safety limit in the clinical scan. A 30 minutes scan was performed. As illustrated by plot 1000 of FIG. 11B, the maximum temperature rise was found as 1.01° C., which is an acceptable temperature increase according to the IEC 60601-2-33 standard.

The present invention has been described in terms of one or more preferred embodiments, and it should be appreciated that many equivalents, alternatives, variations, and modifications, aside from those expressly stated, are possible and within the scope of the invention.

As used in the claims, the phrase “at least one of A, B, and C” or “at least one of A, B, or C” means at least one of A, at least one of B, and/or at least one of C, or any one of A, B, or C or combination of A, B, or C. A, B, and C are elements of a list, and A, B, and C may be anything contained in the Specification.

What is claimed is:

1. A system for transmitting electrical signals to or from the head of a subject, the system comprising:
a support structure configured for placement on a head of a human subject; and
at least one lead system coupled to the support structure, the at least one lead assembly comprising:
a base layer having a top surface, a bottom surface, and extending from a proximal end to a distal end;
an electrical connector attached to a proximal end of the base layer; and
a conductive circuit comprising:
a conductive trace printed as a thin film along the top surface of the base layer and extending between and electrically connecting the electrode and the electrical connector; and
an electrode formed printed on top of the conductive trace at the distal end of the base layer; and
wherein a height of conductive trace as measured from the top surface of the base layer is less than about 50 nm.

2. The system of claim 1, wherein the conductive trace comprises a layer of conductive, non-ferrous material.

3. The system of claim 1, wherein a width of the conductive trace is less than about 200 μm or less than about 100 μm.

4. The system of claim 1, wherein the conductive trace is formed of a plastic film coated with aluminum.

5. The system of claim 1, wherein the electrode is formed as a polymer thick film.

6. The system of claim 1, wherein the height of the conductive trace is about 30 nm or less.

7. The system of claim 1, wherein the electrode comprises a silver foil.

8. The system of claim 1, wherein the silver foil is coated with silver chloride.

9. The system of claim 1, wherein the carrier system includes a plurality of sensor mounts, each sensor mount being configured to engage a corresponding locking mount to secure the distal end of a lead assembly relative to the support structure.

10. The system of claim 9, wherein the carrier assembly includes at least 128 sensor mounts.

11. The system of claim 9, wherein the carrier assembly includes at least 256 sensor mounts, and the at least one lead assembly includes 128 lead systems.

12. A system for transmitting electrical signals to or from the head of a subject, the system comprising:
a base layer having a top surface, a bottom surface, a proximal end, and a distal end;
an electrical connector attached to a proximal end of the base layer; and
a conductive circuit including:
an electrode supported along the top surface of the base layer at the distal end of the base layer; and
a conductive trace supported along the top surface of the base layer, the conductive trace extending between and electrically connecting the electrode and the electrical connector;
wherein the conductive trace is formed from a conductive, non-ferrous material deposited along the upper surface of the base layer using a thin-film deposition process, and the electrode is formed as a polymer thick film printed on the conductive trace at the distal end of the base layer.

13. The system of claim 12, wherein the conductive, non-ferrous material comprises aluminum that has been printed as a thin film along the upper surface of the base layer.

14. The system of claim 14, wherein a height of the conductive trace from the top surface of the base layer is about 30 nm or less.

15. The system of claim 12, wherein the electrode is secured relative to the conductive trace using an electrically conductive adhesive.

16. The system of claim 12, wherein the electrode includes silver coupled to the conductive trace through a conductive adhesive.

17. The system of claim 16, wherein the adhesive includes a carbon-based conductive glue.

18. A method of forming a system for transmitting electrical signals to or from the head of a subject, the method comprising:
providing a base layer;
depositing a layer of conductive, non-ferrous material along an upper surface of the base layer using a thin-film deposition technique;
patterning the deposited non-ferrous material into a conductive trace that extends between a proximal end and a distal end of the base layer; and
electrically coupling a thick film forming an electrode relative to a distal end of the conductive trace.

19. The method of claim 18, wherein the electrode is electrically coupled to the conductive trace using a conductive adhesive.

20. The method of claim 18, further comprising a protective cover layer atop the conductive trace.
